(19) United States
(12) Patent Application Publication
(10) Pub. No.: US 2020/0016283 A1
(43) Pub. Date: Jan. 16, 2020

(54) COMBINATION THERAPY COMPRISING A RADIOPHARMACEUTICAL AND A DNA-REPAIR INHIBITOR

(71) Applicant: Bayer AS, Oslo (NO)

(72) Inventor: Alan CUTHBERTSON, Oslo (NO)

(73) Assignee: Bayer AS, Oslo (NO)

(21) Appl. No.: 16/488,158

(22) PCT Filed: Feb. 22, 2018
(86) PCT No.: PCT/EP2018/054368

§ 371 Date: Aug. 22, 2019

(30) Foreign Application Priority Data
Feb. 24, 2017 (EP) 17157888.3
Mar. 31, 2017 (EP) 17164185.5

(51) Int. Cl. A61K 51/10; A61K 31/5377; A61K 31/519; A61K 31/497; A61P 35/00

(52) U.S. Cl. CPC A61K 51/1027; A61K 31/5377; A61K 31/519; A61K 51/1051; A61P 35/00; A61K 51/103; A61K 51/1072; A61K 31/497

(57) ABSTRACT

The present invention provides a method of combination therapy comprising administration of a tissue-targeting radio-pharmaceutical and a DNA-repair inhibitor. The method may be used in the treatment of hyperplastic or neoplastic disease, such as a carcinoma, sarcoma, myeloma, leukemia, lymphoma or mixed type cancer.

### FILED OF THE INVENTION

The present invention relates to methods of combination therapy for enhancing the efficacy of endo-radiopharmaceutical therapy. The combination therapy of the present invention is in particular useful in the treatment of hyperplastic or neoplastic disease.

### BACKGROUND OF THE INVENTION

Specific cell killing can be essential for the successful treatment of a variety of diseases in mammalian subjects. Typical examples of this are in the treatment of malignant diseases such as sarcomas and carcinomas. However the selective elimination of certain cell types can also play a key role in the treatment of other diseases, especially hyperplastic and neoplastic diseases.

The most common methods of selective treatment are currently surgery, chemotherapy and external beam irradiation. Targeted radionuclide therapy is, however, a promising and developing area with the potential to deliver highly cytotoxic radiation specifically to cell types associated with disease. The most common forms of radiopharmaceuticals currently authorised for use in humans employ beta-emitting and/or gamma-emitting radionuclides. There has, however, been some interest in the use of alpha-emitting radionuclides in therapy because of their potential for more specific cell killing. The radiation range of typical alpha emitters in physiological surroundings is generally less than 100 micrometres, the equivalent of only a few cell diameters. This makes these sources well suited for the treatment of tumours, including micrometastases, because they have the range to reach neighbouring cells within a tumour but if they are well targeted then little of the radiated energy will pass beyond the target cells. Thus, not every cell need be targeted but damage to surrounding healthy tissue may be minimised (see Feinendegen et al., Radiat Res 148:195-201 (1997)). In contrast, a beta particle has a range of 1 mm or more in water (see Wilbur, Antibody Immunocon Radiopharm 4: 85-96 (1991)).

The energy of alpha-particle radiation is high in comparison with that carried by beta particles, gamma rays and X-rays, typically being 5-8 MeV, or 5 to 10 times that of a beta particle and 20 or more times the energy of a gamma ray. Thus, this deposition of a large amount of energy over a very short distance gives α-radiation an exceptionally high linear energy transfer (LET), high relative biological efficacy (RBE) and low oxygen enhancement ratio (OER) compared to gamma and beta radiation (see Hall, “Radiobiology for the radiologist”, Fifth edition, Lippincott Williams & Wilkins, Philadelphia Pa., USA, 2000). This explains the exceptional cytotoxicity of alpha emitting radionuclides and also imposes stringent demands on the biological targeting of such isotopes and upon the level of control and study of alpha emitting radionuclide distribution which is necessary in order to avoid unacceptable side effects.

Several alpha-emitters, such as Terbium-149 (¹⁴⁹Tb), Astatine-211 (²¹¹At), Bismuth-212 (²¹²Bi), Bismuth-213 (²¹³Bi), Actinium-225 (²²⁵Ac), Radium-223 (²²³Ra), Radium-224 (²²⁴Ra), or Thorium-227 (²²⁷Th), have been investigated and/or commercialised for use as radiopharmaceuticals. In particular, the use of ‘tissue-targeting’ radiopharmaceuticals has meant that the radioactive nucleus can be delivered to the target cell (for example a cancerous cell) with an improved accuracy, thus minimising unwanted damage to surrounding tissue and hence minimising side effects. Tissue-targeting radiopharmaceuticals are typically conjugates in which the radiopharmaceutical moiety is linked to a targeting unit, for example via a chelator. The targeting unit (for example, an antibody) guides the radiopharmaceutical to the desired cell (by targeting a particular antigen on a cancer cell for example) such that the alpha radiation can be delivered in close proximity to the target. A small number of elements can be considered “self targeting” due to their inherent properties. Radium, for example, is a calcium analogue and targets bone surfaces by this inherent nature.

One particular class of tissue-targeting radiopharmaceuticals is Targeted Thorium Conjugates (TTCs), in which alpha-emitting thorium-227 (Th-227) nuclei are connected to tumor-targeting moieties such as antibodies. The radioactive pharmaceutical exploits the unique properties of elements that emit alpha particles, and the targeting properties of the conjugates help to minimise undesirable side effects.

Whilst considerable advances have been made over the last few years in the field of targeted radiopharmaceuticals, it would be of considerable advantage to provide further targeted therapeutic methods with increased efficiency. In particular, even with efficient targeting, there is a limit to the amount of radionuclide which can be administered to a subject without causing intolerable side-effects such as myelo-suppression. It would be of considerable benefit to provide a therapeutic method or a method of utilising such radionuclides which could enhance the efficacy of the medicament without requiring a higher dose of radiopharmaceutical.

The present inventors have now established that combinations of targeted radiopharmaceuticals with small molecule DNA-repair inhibitors can improve the therapeutic efficiency of radiopharmaceuticals. In particular, the combination treatment of the present invention may result in an additive, super-additive or synergistic interaction between a radiopharmaceutical and at least one from a range of DNA repair inhibitors and may be employed against various targets and cancer cell lines. A key advantage of the combination therapy of the present invention is the synergistic effect of the DNA repair inhibitor and the tissue-targeting radiopharmaceutical. The DNA repair inhibitor and the tissue-targeting radiopharmaceutical work in tandem to increase the effectiveness in treatment. The combination therapy is thus more effective than the use of the tissue targeting radiopharmaceutical alone or the DNA repair inhibitor alone and the effect of the combination is greater than the sum of the effects of the components used individually.

The synergistic effects of the combination therapies of the present invention have been demonstrated in combination cytotoxicity assays on various cancer cell lines and in in vivo xenograft studies.

### SUMMARY OF THE INVENTION

In a first aspect, the invention provides a method of combination therapy comprising administration of
a) a tissue-targeting radiopharmaceutical, and
b) a DNA-repair inhibitor.

In a particular embodiment, the tissue-targeting radiopharmaceutical comprises an alpha-emitter. In a further particular embodiment, the tissue-targeting radiopharmaceutical is a complex comprising the 4+ ion of an alpha-emitting thorium radionuclide such as Thorium-227. In a further particular embodiment, the tissue-targeting radiopharmaceutical is a targeted thorium conjugate (TTC).

In a further particular embodiment, the tissue-targeting radiopharmaceutical comprises a tissue-targeting moiety selected from a monoclonal or polyclonal antibody, an antibody fragment (such as Fab, F(ab′)2, Fab′ or scFv), a construct of such antibodies and/or fragments, a protein, a peptide or a peptidomimetic. In a further particular embodiment, the tissue-targeting radiopharmaceutical comprises a tissue-targeting moiety which has binding affinity for a target selected from the CD22 receptor, FGFR2, Mesothelin, HER-2, PSMA or CD33, preferably for Mesothelin, FGFR2, HER-2 or CD33, most preferably Mesothelin or FGFR2.

In a further particular embodiment, the DNA-repair inhibitor is an inhibitor of a protein selected from PARP1, ATR, ATM and DNA-PK, preferably ATR.

In a further particular embodiment, the tissue-targeting radiopharmaceutical is administered at a dose level below the level required for a monotherapy response.

In a further particular embodiment, the method is for the treatment of hyperplastic or neoplastic disease, such as a carcinoma, sarcoma, myeloma, leukemia, lymphoma or mixed type cancer, including Non-Hodgkin's Lymphoma or B-cell neoplasms, breast, colorectal, endometrial, gastric, acute myeloid leukemia, prostate or brain, mesothelioma, ovarian, lung or pancreatic cancer.

In a further aspect, the invention provides a tissue-targeting radiopharmaceutical for use in a method of combination therapy for hyperplastic or neoplastic disease, comprising administration of
a) a tissue-targeting radiopharmaceutical, and
b) a DNA-repair inhibitor

simultaneously or sequentially in either order.

In a further aspect, the invention provides a kit containing a tissue-targeting radiopharmaceutical and a DNA-repair inhibitor for simultaneous, separate or sequential use in the treatment of a hyperplastic or neoplastic disease, such as a carcinoma, sarcoma, myeloma, leukemia, lymphoma or mixed type cancer, including Non-Hodgkin's Lymphoma or B-cell neoplasms, breast, colorectal, endometrial, gastric, acute myeloid leukemia, prostate or brain, mesothelioma, ovarian, lung or pancreatic cancer.

In a further aspect, the invention provides a method of treating a hyperplastic or neoplastic disease, such as a carcinoma, sarcoma, myeloma, leukemia, lymphoma or mixed type cancer, including Non-Hodgkin's Lymphoma or B-cell neoplasms, breast, colorectal, endometrial, gastric, acute myeloid leukemia, prostate or brain, mesothelioma, ovarian, lung or pancreatic cancer, comprising administering to an animal, preferably a mammal, e.g. human, effective amounts of the components of a combination therapy as defined herein.

In a further aspect, the invention provides a use of a tissue-targeting radiopharmaceutical in the manufacture of a medicament for the treatment of a hyperplastic or neoplastic disease, such as a carcinoma, sarcoma, myeloma, leukemia, lymphoma or mixed type cancer, including Non-Hodgkin's Lymphoma or B-cell neoplasms, breast, colorectal, endometrial, gastric, acute myeloid leukemia, prostate or brain, mesothelioma, ovarian, lung or pancreatic cancer in a method comprising administration of:
a) a tissue-targeting radiopharmaceutical, and
b) a DNA-repair inhibitor

simultaneously or sequentially in either order.

In a further aspect, the invention provides a kit comprising
a) a tissue-targeting radiopharmaceutical, and
b) a DNA-repair inhibitor.

The features of the aspects and/or embodiments indicated herein are usable individually and in combination in all aspects and embodiments of the invention where technically viable, unless otherwise indicated.

### DETAILED DESCRIPTION OF THE INVENTION

The present invention relates to a combination therapy comprising administration of a tissue targeting radiopharmaceutical and a DNA repair inhibitor. The following discussion, description and definitions apply to all aspects of the present invention, where context allows, unless explicitly indicated otherwise.

### Tissue Targeting Radiopharmaceuticals

In the context of the present invention, “tissue targeting” is used herein to indicate that the substance in question (particularly when in the form of a tissue-targeting complex as described herein), serves to localise itself (and particularly to localise any conjugated thorium complex) preferentially to at least one tissue site at which its presence is desired (e.g. to deliver a radioactive decay). Thus a tissue targeting group or moiety serves to provide greater localisation of a radioisotope to at least one desired site in the body of a subject following administration to that subject in comparison with the concentration of an equivalent radioisotope or complex not bound to the targeting moiety. The targeting moiety in the present case will be preferably selected to bind specifically to cell-surface targets (e.g. receptors) associated with cancer cells or other targets associated with the tumour microenvironment. There are a number of targets which are known to be associated with hyperplastic and neoplastic disease. These include certain receptors, cell surface proteins, transmembrane proteins and proteins/peptides found in the extracellular matrix in the vicinity of diseased cells.

Tissue-targeting radiopharmaceuticals of the various aspects of the present invention preferably comprise a tissue-targeting moiety. Such a moiety may be, for example, an antibody or antibody derivative, such as one selected from a monoclonal or polyclonal antibody, an antibody fragment (such as Fab, F(ab′)2, Fab′. or scFv), or a construct of such antibodies and/or fragments. Mixtures of such antibodies and/or derivatives are evidently also appropriate. Some examples of engineered antibodies are listed herein below.

The targeting moiety is preferably tumour-homing, i.e. it targets cancer cells. Such cancer cell targeting is typically the result of the targeting moiety targeting a tumour-associated antigen. In one embodiment, therefore, the tissue targeting moiety may bind to a tumour-associated antigen. Many such tumour associated antigens are known in the art, including “Cluster of Differentiation (CD)” antigens (e.g. CD20, CD22, CD30, CD32, CD33 and/or CD52), glycoprotein antigens (e.g. EpCAM, CEA, Mucins, TAG-72m Carbonic anhydrase IX, PSMA and/or folate binding protein), Glycolipid antigens (e.g. Gangliosides such as GD2, GD3, and/or GM2), Carbohydrate antigens (e.g. Lewis-Y), Vascular antigens (e.g. VEGF, VEGFR, αVβ3, α5β1), Growth factor antigens (e.g. ErbB1, EGFR, ErbB2, HER2, ErbB3, c-MET, IGF1R, EphA3, TRAIL-R!, TRAIL-R2, RANKL), extracellular matrix antigens (e.g. FAP, Tenascin), and/or overexpressed receptors (e.g αᵥβ₃).

The antibody may be an antibody (e.g. a monoclonal antibody) which is in itself an immunotherapeutic agent which binds to certain cells or proteins and then stimulates the patient's immune system to attack those cells. In this case, the radiopharmaceutical acts in tandem with the immunotherapeutic effects of the antibody. Alternatively, the targeting moiety may act solely as a targeting agent and does not provoke any immunotherapeutic effects by itself. In this case, it is solely the radiopharmaceutical unit which acts as the active, cell-destroying agent, supported in the combination therapy methods of the present invention by at least one DNA repair inhibitor.

In one embodiment, the tissue-targeting radiopharmaceutical may comprise a tissue-targeting moiety selected from at least one engineered antibody. Such an engineered antibody may be an antibody that comprises an epitope binding domain (for example, but not limited to, an antibody variable region having all 6 CDRs, or an equivalent region that is at least 90% identical to an antibody variable region) chosen from: abagovomab, abatacept (also known as ORENCIA®), abciximab (also known as REOPRO®, c7E3 Fab), adalimumab (also known as HUMIRA®), adecatumumab, alemtuzumab (also known as CAMPATH®, MabCampath or Campath-1H), altumomab, afelimomab, anatumomab mafenatox, anetumumab, anrukizumab, apolizumab, arcitumomab, aselizumab, atlizumab, atorolimumab, bapineuzumab, basiliximab (also known as SIMULECT®), bavituximab, bectumomab (also known as LYMPHOSCAN®), belimumab (also known as LYMPHO-STAT-B®), bertilimumab, besilesomab, bevacizumab (also known as AVASTIN®), biciromab brallobarbital, bivatuzumab mertansine, campath, canakinumab (also known as ACZ885), cantuzumab mertansine, capromab (also known as PROSTASCINT®), catumaxomab (also known as REMOVAB®), cedelizumab (also known as CIMZIA®), certolizumab pegol, cetuximab (also known as ERBITUX®), clenoliximab, dacetuzumab, dacliximab, daclizumab (also known as ZENAPAX®), denosumab (also known as AMG 162), detumomab, dorlimomab aritox, dorlixizumab, duntumumab, durimulumab, durmulumab, ecromeximab, eculizumab (also known as SOLIRIS®), edobacomab, edrecolomab (also known as Mab17-1A, PANOREX®), efalizumab (also known as RAPTIVA®), efungumab (also known as MYCOGRAB®), elsilimomab, enlimomab pegol, epitumomab cituxetan, efalizumab, epitumomab, epratuzumab, erlizumab, ertumaxomab (also known as REXOMUN®), etanercept (also known as ENBREL®), etaracizumab (also known as etaratuzumab, VITAXIN®, ABEGRIN™), exbivirumab, fanolesomab (also known as NEUTROSPEC®), faralimomab, felvizumab, fontolizumab (also known as HUZAF®), galiximab, gantenerumab, gavilimomab (also known as ABX-CBL®), gemtuzumab ozogamicin (also known as MYLOTARG®), golimumab (also known as CNTO 148), gomiliximab, ibalizumab (also known as TNX-355), ibritumomab tiuxetan (also known as ZEVALIN®), igovomab, imciromab, infliximab (also known as REMICADE®), inolimomab, inotuzumab ozogamicin, ipilimumab (also known as MDX-010, MDX-101), iratumumab, keliximab, labetuzumab, lemalesomab, lebrilizumab, lerdelimumab, lexatumumab (also known as, HGS-ETR2, ETR2-ST01), lexitumumab, libivirumab, lintuzumab, lucatumumab, lumiliximab, mapatumumab (also known as HGS-ETR1, TRM-1), maslimomab, matuzumab (also known as EMD72000), mepolizumab (also known as BOSATRIA®), metelimumab, milatuzumab, minretumomab, mitumomab, morolimumab, motavizumab (also known as NUMAX™), muromonab (also known as OKT3), nacolomab tafenatox, naptumomab estafenatox, natalizumab (also known as TYSABRI®, ANTEGREN®), nebacumab, nerelimomab, nimotuzumab (also known as THERACIM hR3®, THERA-CIM-hR3®, THERALOC®), nofetumomab merpentan (also known as VERLUMA®), ocrelizumab, odulimomab, ofatumumab, omalizumab (also known as XOLAIR®), oregovomab (also known as OVAREX®), otelixizumab, pagibaximab, palivizumab (also known as SYNAGIS®), panitumumab (also known as ABX-EGF, VECTIBIX®), pascolizumab, pemtumomab (also known as THERAGYN®), pertuzumab (also known as 2C4, OMNITARG®), pexelizumab, pintumomab, priliximab, pritumumab, ranibizumab (also known as LUCENTIS®), raxibacumab, regavirumab, reslizumab, rituximab (also known as RITUXAN®, MabTHERA®), rovelizumab, ruplizumab, satumomab, sevirumab, sibrotuzumab, siplizumab (also known as MEDI-507), sontuzumab, stamulumab (also known as MYO-029), sulesomab (also known as LEUKOSCAN®), tacatuzumab tetraxetan, tadocizumab, talizumab, taplitumomab paptox, tefibazumab (also known as AUREXIS®), telimomab aritox, teneliximab, teplizumab, ticilimumab, tocilizumab (also known as ACTEMRA®), toralizumab, tositumomab, trastuzumab (also known as HERCEPTIN®), tremelimumab (also known as CP-675,206), tucotuzumab celmoleukin, tuvirumab, urtoxazumab, ustekinumab (also known as ONTO 1275), vapaliximab, veltuzumab, vepalimomab, visilizumab (also known as NUVION®), volociximab (also known as M200), votumumab (also known as HUMASPECT®), zalutumumab, zanolimumab (also known as HuMAX-CD4), ziralimumab, or zolimomab aritox.

Whilst antibodies as tissue-targeting moiety constitute a preferred embodiment of the invention, the targeting unit may also be a single type of protein, protein fragment or construct of protein, or a mixture of proteins, fragments or constructs of protein. Where peptides are referred to herein, corresponding peptidomimetics may also be utilised. Combinations of targeting moieties of any type may also be used.

The targeting moiety may also be a peptide such as Tat-peptide, penetratin, MPG and Pep-1. Protein fragments, such as histidine-rich glycoprotein fragments, for example HRGP-335 also constitute an embodiment of the invention. Tumor-homing peptides such as the NGR- and cRGD peptides constitute a further embodiment. Suitable moieties also include other poly- and oligo-peptides including peptidomemetics.

The targeting moiety may also be a small molecule ligand. By small molecule ligand is meant a ligand of low molecular weight, for example having a molecular weight of less than 1000 g/mol (e.g. 50 to 1000), preferably less than 500 or less than 250 g/mol. In particular, the targeting moiety may be a PSMA-targeting ligand. Of particular interest are ligands targeting the enzymatic binding pocket derived from either phosphonate, phosphate and phosphoramidates, thiols and ureas. Suitable PSMA ligands may, for example, comprise at least one moiety selected from a carbon-sulfur double bond, a phosphorus-sulfur double bond, a phosphorus-sulfur single bond, a thioester, a phosphonate, a phosphate, a phosphoramidate, a thiol, and/or a urea.

It is also envisaged that aptamers, DNA or RNA fragments may be used as targeting moieties in the present invention.

Surface-modified nanoparticles that include, but are not limited to, liposomes, nanoworms, and dendrimers may also be used as the targeting unit and thus constitute a further embodiment of the invention.

Examples of cell-surface receptors and antigens which may be associated with neoplastic disease include CD22, CD33, FGFR2 (CD332), PSMA, HER2, Mesothelin etc. Therefore, in a particularly preferred embodiment of the invention, the tissue-targeting moiety (e.g. peptide or protein) has specificity for at least one antigen or receptor selected from CD22, CD33, FGFR2 (CD332), PSMA, HER2 and Mesothelin.

CD22, or cluster of differentiation-22, is a molecule belonging to the SIGLEC family of lectins (SIGLEC=Sialic acid-binding immunoglobulin-type lectins). CD33 or Siglec-3 is a transmembrane receptor expressed on cells of myeloid lineage. FGFR2 is a receptor for fibroblast growth factor. It is a protein that in humans is encoded by the FGFR2 gene residing on chromosome 10. HER2 is a member of the human epidermal growth factor receptor (HER/EGFR/ERBB) family. Prostate-specific membrane antigen (PSMA) is an enzyme that in humans is encoded by the FOLH1 (folate hydrolase 1) gene. Mesothelin, also known as MSLN, is a protein that in humans is encoded by the MSLN gene.

A particularly preferred tissue-targeting binder in the present case will be selected to bind specifically to CD22 receptor. This may be reflected, for example by having 50 or more times greater binding affinity for cells expressing CD22 than for non-CD22 expressing cells (e.g. at least 100 time greater, preferably at least 300 times greater). It is believed that CD22 is expressed and/or over-expressed in cells having certain disease states (as indicated herein) and thus the CD22 specific binder may serve to target the complex to such disease-affected cells. Similarly a tissue targeting moiety may bind to cell-surface markers (e.g. CD22 receptors) present on cells in the vicinity of disease affected cells. CD22 cell-surface markers may be more heavily expressed on diseased cell surfaces than on healthy cell surfaces or more heavily expressed on cell surfaces during periods of growth or replication than during dormant phases. In one embodiment, a CD22 specific tissue-targeting binder may be used in combination with another binder for a disease-specific cell-surface marker, thus giving a dual-binding complex. Tissue-targeting binders for CD-22 will typically be peptides or proteins, as discussed herein. The various aspects of the invention as described herein relate to treatment of disease, particularly for the selective targeting of diseased tissue, as well as relating to complexes, conjugates, medicaments, formulation, kits etc. useful in such methods. In all aspects, the diseased tissue may reside at a single site in the body (for example in the case of a localised solid tumour) or may reside at a plurality of sites (for example where several joints are affected in arthritis or in the case of a distributed or metastasised cancerous disease).

Other ligands particularly suitable for various embodiments applicable to all aspects of the invention include PSMA ligands for use in prostate cancer, HER2 ligands for use in breast and gastric cancer, and Mesothelin ligands for use in mesothelioma, ovarian, lung and pancreatic cancers. Suitable ligands/binders for each of these targets are known in the art and may be applied using the methods described herein.

### Radioactive Nuclei

The tissue-targeting radiopharmaceutical preferably comprises an alpha-emitter. The radioactive isotope may be any alpha-emitting isotope (i.e. an alpha emitter) suitable for use in the treatments of the present invention. The alpha emitters may be selected from the group consisting of Terbium-149 (¹⁴⁹Tb), Astatine-211 (²¹¹At), Bismuth-212 (²¹²Bi), Bismuth-213 (²¹³Bi), Actinium-225 (²²⁵Ac), or Thorium-227 (²²⁷Th). Preferably, the alpha-emitting nucleus is Thorium-227.

In one embodiment of the present invention, the alpha-emitting radioisotope is not Radium 223 (²²³Ra) or Radium-224 (²²⁴Ra) It is particularly preferable that the alpha-emitting radioisotope is not Radium-223 (²²³Ra). In such an embodiment, it is preferred that the radiopharmaceutical comprises an alpha-emitting radioisotope other than Radium-223. In a corresponding embodiment, the radiopharmaceutical does not comprise any Radium-223 or includes ²²³Ra only as a decay product and/or unavoidable impurity. In a further embodiment, it is preferably if the alpha-emitting radioisotope can be complexed and/or conjugated to ligands.

In a particular embodiment of the invention the tissue-targeting radiopharmaceutical is a complex comprising the 4+ ion of an alpha emitting thorium radionuclide, such as Thorium-227. Preferably, the tissue-targeting radiopharmaceutical is a targeted thorium conjugate (TTC). The targeted thorium conjugate may be any conjugate which comprises an alpha-radioactive thorium ion (e.g. Thorium-227 ion) linked to a targeting moiety such as those described previously. In particular, preferred targeted thorium conjugates include MSLN-TTC, FGFR2-TTC, HER2-TTC, PSMA-TTC, and CD33-TTC.

In one embodiment, MSLN-TTC is BAY2287411 and is prepared according to Example 7, specifically Examples 7a and 7b of WO 2016/096843.

In one embodiment, FGFR2-TTC is BAY2304058 and is prepared according to Example 6, specifically Examples 6a and 6b of WO 2016/096843.

In one embodiment, HER2-TTC is BAY 2331370 and is prepared according to Example 5, specifically Examples 5a and 5b of WO 2016/096843.

In one embodiment, PSMA-TTC is BAY 2315497 and is prepared according to Example 9, specifically Examples 9a and 9b of WO 2016/096843. The monoclonal antibody may be AB-PG1-XG1-006 as disclosed in WO 03/034903.

Radioactive thorium-containing compounds (e.g. comprising Th-227) may be used in high dose regimens, where the myelotoxicity of the generated radium (e.g. Ra-223) would normally be intolerable, when stem cell support or a comparable recovery method is included. Without supportive intervention, the maximum dose of a nuclide such as ²²⁷Th may be limited by such myelotoxicity and might be stopped, for example, to avoid depressing the the neutrophil cell count below 20% or 10% of its initial value at nadir. In cases of stem-cell support or similar supportive therapy is provided, the neutrophil cell count may be reduced to below 10% at nadir and exceptionally will be reduced to 5% or if necessary below 5%, providing suitable precautions are taken and subsequent stem cell support is given. Such techniques are well known in the art.

Alpha-emitting thorium is the preferred radioactive element comprised in the tissue-targeting radiopharmaceuticals referred to herein and Thorium-227 is the preferred isotope for all references to thorium herein where context allows. Thorium-227 is relatively easy to produce and can be prepared indirectly from neutron irradiated Ra-226, which will contain the mother nuclide of Th-227, i.e. Ac-227 (T1/2=22 years). Actinium-227 can quite easily be separated from the Ra-226 target and used as a generator for Th-227. This process can be scaled to industrial scale if necessary, and hence the supply problem seen with most other alpha-emitters considered candidates for molecular targeted radiotherapy can be avoided. Thorium-227 decays via radium-223. In this case the primary daughter has a half-life of 11.4 days. From a pure Th-227 source, only moderate amounts of radium are produced during the first few days. However, the potential toxicity of Ra-223 is higher than that of Th-227 since the emission from Ra-223 of an alpha particle is followed within minutes by three further alpha particles from the short-lived daughters.

Partly because it generates potentially harmful decay products, thorium-227 (T1/2=18.7 days) has not been widely considered for alpha particle therapy.

Thorium-227 may be administered in amounts sufficient to provide desirable therapeutic effects without generating so much radium-223 as to cause intolerable bone marrow suppression. It is desirable to maintain the daughter isotopes in the targeted region so that further therapeutic effects may be derived from their decay. However, it is not necessary to maintain control of the thorium decay products in order to have a useful therapeutic effect without inducing unacceptable myelotoxicity. Without being bound by theory, this is believed to be because at least partial incorporation of the radium-223 into bone and the short half-life of the daughters serves to titrate the potentially harmful daughter nuclei away from sensitive structures such as the bone marrow.

The alpha-emitting isotope of the radiopharmaceutical may be linked to the tissue-targeting moiety via any suitable ligand. Such a ligand will be selected to be appropriate for the chemistry of the relevant element and oxidation state and suitable chelators are generally well-known in the art.

Previously known chelators for thorium, for example, include the polyaminopolyacid chelators which comprise a linear, cyclic or branched polyazaalkane backbone with acidic (e.g. carboxyalkyl) groups attached at backbone nitrogens. Examples of such chelators include DOTA derivatives such as p-isothiocyanatobenzyl-1,4,7,10-tetraazacyclododecane-1,4,7,10-tetraacetic acid (p-SCN-Bz-DOTA) and DTPA derivatives such as p-isothiocyanatobenzyl-diethylenetriaminepentaacetic acid (p-SCN-Bz-DTPA), the first being cyclic chelators, the latter linear chelators.

In one particular embodiment of the invention, the tissue-targeting radiopharmaceutical comprises a tissue-targeting moiety covalently bound to an octadentate ligand, examples of which include ligands comprising at least one 3,2- hydroxypyridinone (3,2-HOPO) moiety. Said ligand may be complexed to a 4+ metal ion such as that of and alpha-emitting thorium radionuclide (e.g. ²²⁷Th). Such ligands are described, for example, in WO2011/098611 which is incorporated herein by reference. The ligand may therefore be an octadentate ligand, particularly an octadentate hydroxypyridinone-containing ligand. Such ligands will typically comprise at least one chelating group of the following substituted pyridine structure (I):

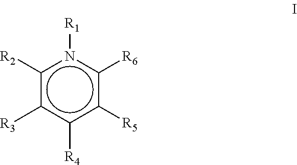

Wherein R₁ is an optional N-substituent group and may thus be absent or may be selected from hydrocarbyl, OH, O-hydrocarbyl, SH and S-hydrocarbyl groups (e.g. methyl or ethyl); comprises a linker moiety; and/or comprises a coupling moiety; groups R₂ to R₆ are each independently selected from H, OH, ═O, short hydrocarbyl groups (e.g. methyl, ethyl, propyl), linker moieties (linking to other moieties of formula I) and/or coupling moieties (coupling to targeting agents). Favoured ligands may have four moieties of formula I as described in WO2011/098611. Particular examples include octadentate 3,2-HOPO ligands such as those indicated below, as well as equivalent ligands additionally substituted with linker groups (if needed), as discussed herein:

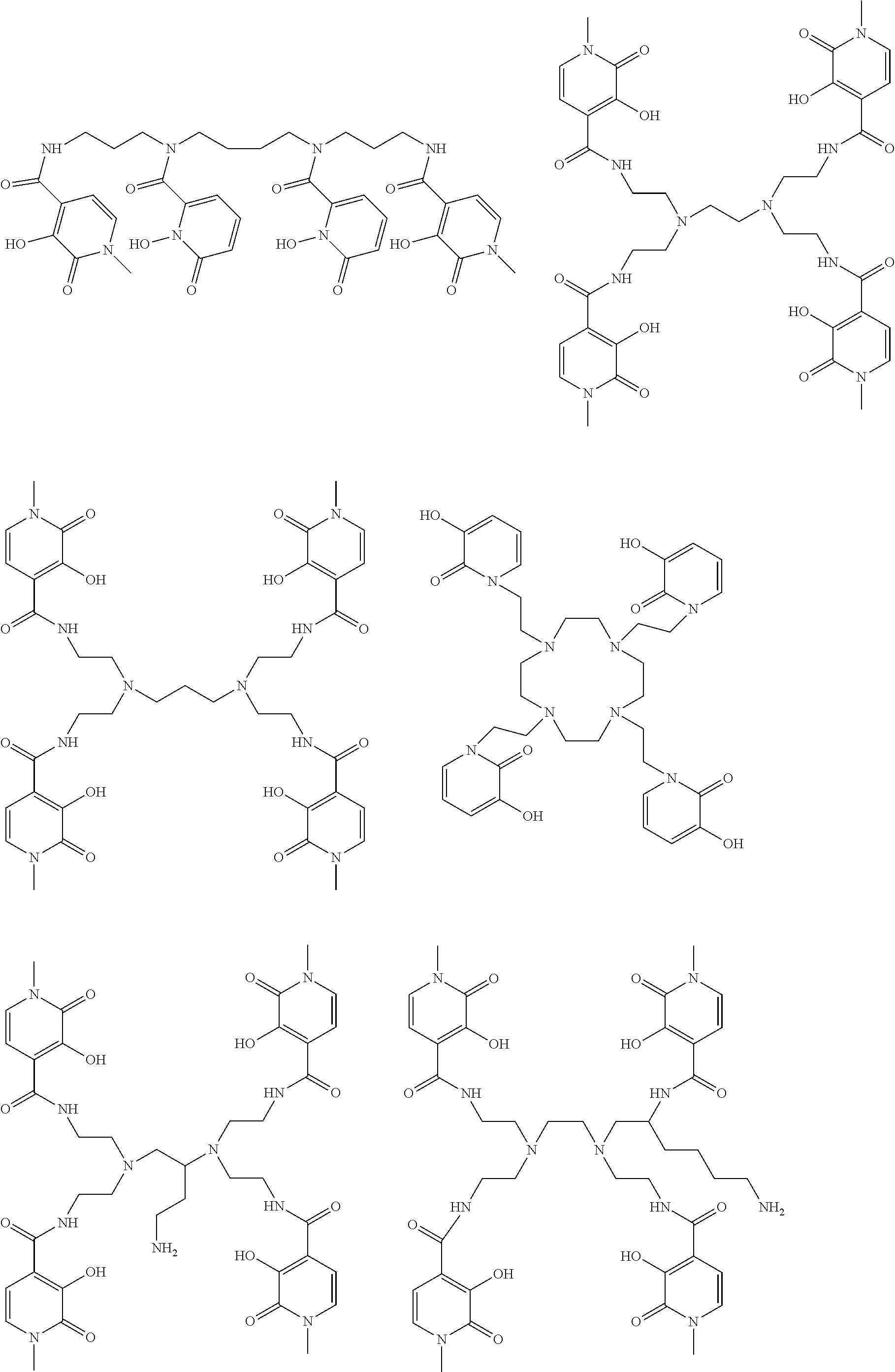

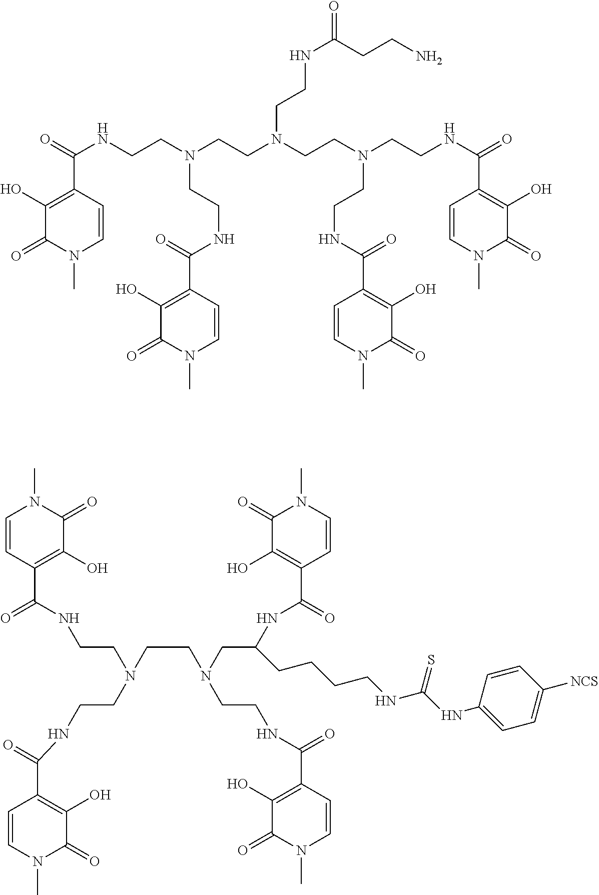

An alternative favoured embodiment utilises ligands as described in WO2013/167756, which is incorporated herein by reference. Such ligands may also be complexed to a 4+ metal ion such as that of an alpha-emitting thorium radionuclide (e.g. ²²⁷Th). In such a particular embodiment, the ligand can be an octadentate ligand comprising at least one and preferably two or four chelating moieties of formula II:

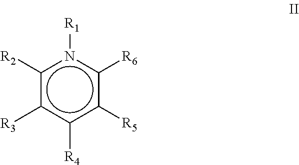

Wherein R₁ is an optional N-substituent solubilising group which will be present in at least one of the moieties of formula II (e.g. in 1 to 4 of four moieties of formula II) and comprises a hydroxyalkyl group (e.g. hydroxymethyl or hydroxydethyl group); groups R₂ to R₆ are each independently selected from H, OH, ═O, short hydrocarbyl groups, linker moieties and/or coupling moieties wherein one of R₂ to R₆ is OH and one of R₂ to R₆ is ═O. The remaining groups R₂ to R₆ may be as described above. The ligand may for example be a ligand of structure III:

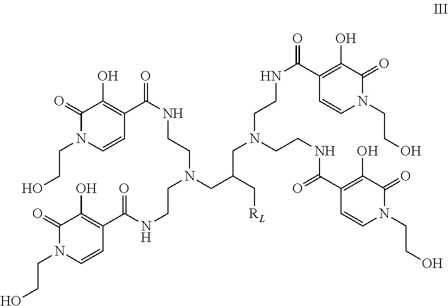

Wherein R_{L} is any suitable linker moiety such as —Ph-NH₂, —Ph-NCS, —Ph-NH—CO—C₂H₄—CO₂H or any described herein.

As used herein, the term “linker moiety” is used to indicate a chemical entity which serves to join at least two chelating groups in the octadentate ligands, which form a key component in various aspects of the invention. Typically, each chelating group (e.g. those of formula I above and/or formula II below) will be bi-dentate and so four chelating groups, of which at least one is of formula I, will typically be present in the ligand. Such chelating groups are joined to each other by means of their linker moieties. Thus, a linker moiety (as used above) may be shared between more than one chelating group of formula I and/or II. The linker moieties may also serve as the point of attachment between the complexing part and the targeting moiety. In such a case, at least one linker moiety will join to a coupling moiety (see below). Suitable linker moieties include short hydrocarbyl groups, such as C1 to C12 hydrocarbyl, including C1 to C12 alkyl, alkenyl or alkynyl group, including methyl, ethyl, propyl, butyl, pentyl and/or hexyl groups of all topologies.

Linker moieties may also be or comprise any other suitably robust chemical linkages including esters, ethers, amine and/or amide groups. The total number of atoms joining two chelating moieties (counting by the shortest path if more than one path exists) will generally be limited, so as to constrain the chelating moieties in a suitable arrangement for complex formation. Thus, linker moieties will typically be chosen to provide no more than 15 atoms between chelating moieties, preferably, 1 to 12 atoms, and more preferably 1 to 10 atoms between chelating moieties. Where a linker moiety joins two chelating moieties directly, the linker will typically be 1 to 12 atoms in length, preferably 2 to 10 (such as ethyl, propyl, n-butyl etc). Where the linker moiety joins to a central template (see below) then each linker may be shorter with two separate linkers joining the chelating moieties. A linker length of 1 to 8 atoms, preferably 1 to 6 atoms may be preferred in this case (methyl, ethyl and propyl being suitable, as are groups such as these having an ester, ether or amide linkage at one end or both).

A “coupling moiety” as used herein serves to link the ligand component (e.g. with 4 moieties of formula I and/or II) to the targeting moiety. Preferably coupling moieties will be covalently linked to the chelating groups, either by direct covalent attachment to one of the chelating groups or more typically by attachment to a linker moiety or template. Should two or more coupling moieties be used, each can be attached to any of the available sites such as on any template, linker or chelating group.

In one embodiment, the coupling moiety may have the structure:

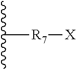

wherein R₇ is a bridging moiety, which is a member selected from substituted or unsubstituted alkyl, substituted or unsubstituted heteroalkyl, substituted or unsubstituted heterocycloalkyl, substituted or unsubstituted aryl and substituted or unsubstituted heteroaryl; and X is a targeting moiety or a reactive functional group. The preferred bridging moieties include all those groups indicated herein as suitable linker moieties. Preferred targeting moieties include all of those described herein and preferred reactive X groups include any group capable of forming a covalent linkage to a targeting moiety, including, for example, COOH, OH, SH, NHR and COH groups, where the R of NHR may be H or any of the short hydrocarbyl groups described herein. Highly preferred groups for attachment onto the targeting moiety include epsilon-amines of lysine residues and thiol groups of cysteine residues. Non-limiting examples of suitable reactive X groups, include N-hydroxysuccimidylesters, imidoesters, acylhalides, N-maleimides, alpha-halo acetyl and isothiocyanates, where the latter three are suitable for reaction with a thiol group.

Another typical example of an octadentate chelator suitable for use in the present invention is the compound of formula IV below, which utilises the 3-hydroxy-N-methyl-2-pyridinone moiety, abbreviated as Me-3,2-HOPO.

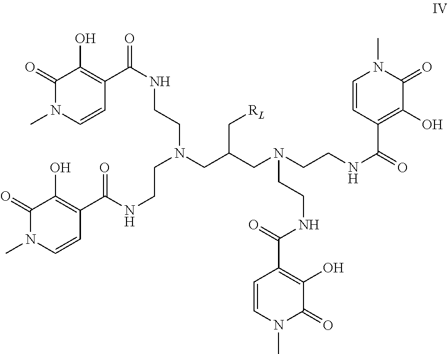

In a particularly favoured embodiment, R_{L} may be such that formula IV is the compound of formula IV′:

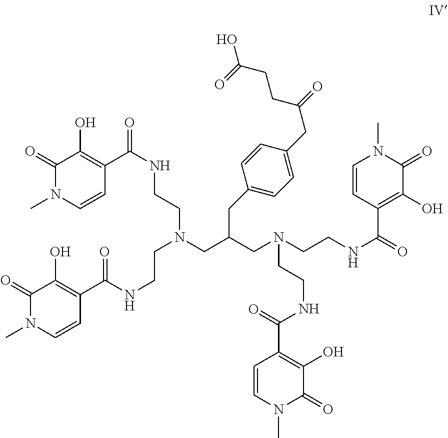 [SMILES: Cn1ccc(C(=O)NCCN(CCNC(=O)c2ccn(C)c(=O)c2O)CC(Cc2ccc(CC(=O)CCC(=O)O)cc2)CN(CCNC(=O)c2ccn(C)c(=O)c2O)CCNC(=O)c2ccn(C)c(=O)c2O)c(O)c1=O]

This particular chelator (IV′) has been found to complex Th-227 in near quantitative yield at ambient temperature in aqueous solutions, and the resulting complexes are highly stable. The carboxylic acid group facilitates conjugation to biomolecules such as antibodies. The synthesis, labelling and in vivo distribution in mice are described in: Bioorganic & Medicinal Chemistry Letters 26 (2016) 4318-4321. It has been shown that the above compound IV′ outperforms 1,4,7,10-tetraazacycloododecane-N, N′,N″,N″′-tetraacetic acid (DOTA) in Th-227 complexation.

### DNA Repair Inhibitor

All aspects of the present invention relate to a combination therapy involving the administration of a tissue targeting radiopharmaceutical and a DNA repair inhibitor. The DNA repair inhibitor utilised in the present invention may be an inhibitor of key proteins of single and/or double strand DNA repair. Mixtures of DNA repair inhibitors may also be utilised.

In a particular embodiment of the invention, the DNA repair inhibitor is selected from the group consisting of inhibitors of PARP1, ATR, ATM and DNA-PK. In one preferred embodiment, the DNA repair inhibitor is an ATR inhibitor. The inhibitor molecules are abbreviated herein by the use of a lower case ‘i’ behind the target protein, e.g. ATRi, ATMi etc.

Without being bound by theory, it is believed that the DNA repair inhibitor sensitizes the target cell to the effects of the alpha radiation. Administration of a DNA repair inhibitor results in the target cell becoming more sensitive to DNA damage caused by the alpha emitter due to a reduced ability to repair that damage, and/or arrest the cell-cycle while such damage is repaired; the cell-destroying efficiency of the tissue-targeting radiopharmaceutical is therefore increased. Since DNA damage can be incurred at any time and rapidly dividing cells such as cancer cells may be particularly prone to such damage, DNA repair inhibitors may have utility when used alone in cancer therapy. The present inventors have, however, observed a synergistic effect by the combination of the DNA repair inhibitor and the tissue-targeting radiopharmaceutical. This effect is greater than the sum of the individual effects exhibited by the DNA repair inhibitor and the tissue-targeting radiopharmaceutical when used separately. Such a synergistic effect is highly desirable for increasing pharmaceutical efficacy.

ATR inhibitors are highly suitable DNA repair inhibitors for use in the various aspects of the present invention. These have previously been reported to sensitize cells to DNA damaging agents (Fokas, E., et al. Cancer Treat Rev, 2014. 40(1): p. 109-17). ATR inhibitors are believed to target the ATR kinase, which is a key protein in late G2 phase arrest and DNA repair. It is activated by DNA damage and will further activate the downstream protein Chk1 by phosphorylation, resulting in arrest and initiation of repair. As most cancer cells are defect in G1 phase of the cell cycle they are often dependent on G2 arrest for the repair of DNA. When G2 arrest is suppressed the cell will continue with mitosis without repair of damage, which may eventually lead to mitotic catastrophe.

ATM serine/threonine kinase, symbolised as ATM, is a serine/threonine kinase that is recruited and activated by DNA double-strand breaks. It phosphorylates several key proteins that initiate activation of the DNA damage checkpoint, leading to cell cycle arrest, DNA repair or apoptosis.

PARP1 has a role in repair of single-stranded DNA (ssDNA) breaks. PARP1 works by modifying nuclear proteins by poly ADP-ribosylation. It also works in conjunction with BRCA, which acts on double strands; members of the PARP family act on single strands; or, when BRCA fails, PARP can takes over those jobs as well (in a DNA repair context).

The DNA repair inhibitor is preferably a small molecule selected from the group consisting of analogues derived from ATR inhibitors including but not limited to BAY1895344, Schisandrin B, NU6027, NVP-BEZ235, VX-803, VX-970, VE-821, VE-822, AZ20, AZD6738; ATM inhibitors including but not limited to AZD0156, Wortmannin, CP-466722, KU-55933, KU-60019, KU-559403, as described in Pharmacology and Therapeutics 149 (2015) 124-138; and DNA-PK inhibitors including but not limited to VX984 PI-103, NU7441, PIK-75, NU7026, PP121, CC-115 and KU-0060648.

In a preferred embodiment, the ATR inhibitor of the combination therapy of the present invention is 2-[(3R)-3-methylmorpholin-4-yl]-4-(1-methyl-1H-pyrazol-5-yl)-8-(1H-pyrazol-5-yl)-1,7-naphthyridine (BAY1895344), or a stereoisomer, a tautomer, an N-oxide, a hydrate, a solvate, or a pharmaceutically acceptable salt thereof.

In another preferred embodiment, the ATR inhibitor of the combination therapy of the present invention is Compound A of structure

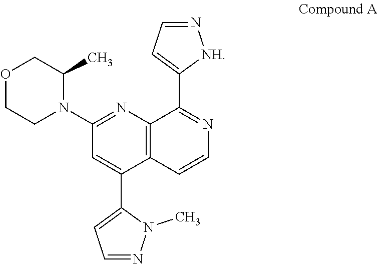 [SMILES: C[C@@H]1COCCN1c1cc(-c2ccnn2C)c2ccnc(-c3ccn[nH]3)c2n1]

The synthesis of Compound A is described in Example 111 of WO2016020320 (A1) and Compound A is referred to in the Examples as BAY 1895344.

In context with the present invention the term “VX-803” is understood as meaning 2-amino-6-fluoro-N-[5-fluoro-4-(4-{[4-(oxetan-3-yl)piperazin-1-yl]carbonyl}piperidin-1-yl)pyridin-3-yl]pyrazolo[1,5-a]pyrimidine-3-carboxamide.

In another embodiment, the ATR inhibitor is VX-803 of structure

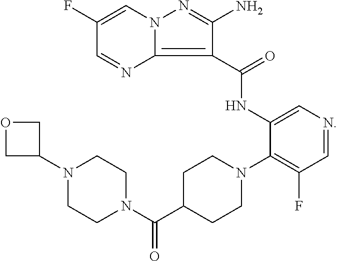 [SMILES: Nc1nn2cc(F)cnc2c1C(=O)Nc1cncc(F)c1N1CCC(C(=O)N2CCN(C3COC3)CC2)CC1]

In context with the present invention the term “VX-970” is understood as meaning 3-(3-{4-[(methylamino)methyl]phenyl}-1,2-oxazol-5-yl)-5-[4-(propan-2-ylsulfonyl)phenyl]pyrazin-2-amine.

In another embodiment, the ATR inhibitor is VX-970 of structure

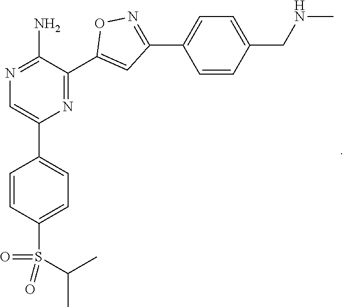 [SMILES: CNCc1ccc(-c2cc(-c3nc(-c4ccc(S(=O)(=O)C(C)C)cc4)cnc3N)on2)cc1 ; "."]

In context with the present invention the term “AZD-6738” is understood as meaning 4-{4-[(3R)-3-methylmorpholin-4-yl]-6-[1-(S-methylsulfonimidoyl)cyclopropyl]pyrimidin-2-yl}-1H-pyrrolo[2,3-b]pyridine.

In another embodiment, the ATR inhibitor is AZD-6738 of structure

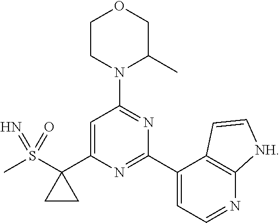 [SMILES: CC1COCCN1c1cc(C2(S(C)(=N)=O)CC2)nc(-c2ccnc3[nH]ccc23)n1]

Examples of preferable FDA-approved PARP inhibitors include Olaparib and Rucaparib. Other examples of PARP inhibitors suitable for the present invention include: Niraparib, Iniparib, Talazoparib, Veliparib, Rucaparib, CEP-9722, Eisai's E7016, BGB-290 and 3-aminobenzamide.

The combination of a TTC with an ATRi is highly preferred. Without being bound by theory, it is believed from cellular mechanistic assays of TTC and ATRi combinations, that ATRi suppresses TTC-induced ATR kinase signalling, suppresses TTC-induced G2-cell cycle arrest and suppresses repair of double strand DNA break.

The DNA repair inhibitors of the present invention may also be DNA repair inhibitors which inhibit proteins which are upstream or downstream from PARP1, ATR, ATM and DNA-PK in the known biochemical pathways for DNA repair (for example, as shown in FIG. 1). In a particular embodiment of the present invention, the DNA repair inhibitor may be a PI3k inhibitor, an EGFR inhibitor and/or antibody, an AKT inhibitor, an mTOR inhibitor, an MEK inhibitor, a WEE1 inhibitor, a Chk1 and/or Chk2 inhibitor, or a RAD51 inhibitor. In a preferred embodiment, the DNA repair inhibitor is a PI3k inhibitor or an EGFR inhibitor and/or antibody. Some of the inhibitors are closely related to the PARP1, ATR, ATM and DNA-PK proteins; for example, Chk1 and Chk2 are directly downstream of ATR and ATM, respectively (see FIG. 1). It is envisaged that inhibitors which work upstream or downstream (directly or indirectly) from any of the inhibitors discussed (especially PARP1i, ATRi, ATMi and DNA-PKi) will also provide beneficial synergistic effects when combined with the tissue-targeting radiopharmaceuticals of the invention. Preferred combinations of inhibitors therefore include at least two inhibitors which function on the same pathway. Examples include ATR with Chk1 and ATM with Chk2.

Examples of PI3k inhibitors which are within the scope of the invention include BKM120, BYL719, CAL-101, GDC-0941, PX-866 and XL147. Examples of EGFR inhibitors/antibodies which are within the scope of the invention include Cetuximab, Tarceva and Gefitinib. Examples of AKT inhibitors which are within the scope of the invention include GSK2141795, MK-2206, Perifosine and SR_{13668.} Examples of mTOR inhibitors which are within the scope of the invention include AZD2014, AZD8055, CC-223, RAD001, MK-8669, Rapamycin and CC1-779. Examples of MEK inhibitors which are within the scope of the invention include ARRY-162, AZD8330, BAY 86-9766, RO_{4987655,} AZD6244 and TAK-733. An example of a WEE1 inhibitor which is within the scope of the invention is AZD1775. Examples of Chk1/Chk2 inhibitors which are within the scope of the invention include MK-8776, PF-477736 and AZD7762. An example of a RAD51 inhibitor which is within the scope of the invention is B02. In a particularly preferred embodiment, the DNA repair inhibitor is Cetuximab.

It is within the scope of the invention that a combination of two or more DNA repair inhibitors be used. In a particular embodiment, two or more inhibitors which inhibit proteins which are downstream/upstream of each other may be used, i.e. two or more DNA inhibitors may be used to target two or more proteins in the same biochemical pathway (for example, as presented in FIG. 1). In a further embodiment, two or more DNA repair inhibitors may be used which target proteins in different biochemical DNA repair pathways. For example, one or more DNA repair inhibitors which target(s) proteins associated with the repair of single-strand breaks may be used with one or more inhibitors targeting proteins associated with the repair of double-strand breaks. Further combinations with DNA repair inhibitors of interstrand crosslink repair, intrastrand crosslink repair, base mismatch repair and/or base modification repair are also envisaged.

### Administration

The tissue-targeting radiopharmaceutical and the DNA repair inhibitor may be administered sequentially in either order, or simultaneously. In a particular embodiment, the tissue-targeting radiopharmaceutical and the DNA repair inhibitor are administered sequentially in either order. In a further particular embodiment, the tissue-targeting pharmaceutical is administered before the DNA-repair inhibitor. In this case, the DNA-repair inhibitor is preferably administered at least two days after administration of the tissue-targeting radiopharmaceutical, such as 2-15 days, preferably 4-10 days, more preferably 6-8 days. For example, the DNA repair inhibitor may be administered 7 days after the administration of the tissue-targeting radiopharmaceutical.

In all aspects of the present invention, the tissue-targeting radiopharmaceutical preferably comprises Th-227. The radiopharmaceutical is preferably administered at a dosage level of thorium-227 dosage of 18 to 400 kBq/kg bodyweight, preferably 20 to 200 kBq/kg, (such as 50 to 200 kBq/kg) more preferably 75 to 170 kBq/kg, especially 100 to 130 kBq/kg. Correspondingly, a single dosage until may comprise around any of these ranges multiplied by a suitable bodyweight, such as 30 to 150 Kg, preferably 40 to 100 Kg (e.g. a range of 540 kBq to 4000 KBq per dose etc). The thorium dosage, the complexing agent and the administration route will moreover desirably be such that the radium-223 dosage generated in vivo is less than 300 kBq/kg, more preferably less than 200 kBq/kg, still more preferably less than 150 kBq/kg, especially less than 100 kBq/kg. Again, this will provide an exposure to Ra-223 indicated by multiplying these ranges by any of the bodyweights indicated. The above dose levels are preferably the fully retained dose of Th-227 but may be the administered dose taking into account that some Th-227 will be cleared from the body before it decays.

Where the biological half-life of the Th-227 complex is short compared to the physical half-life (e.g. less than 7 days, especially less than 3 days) significantly larger administered doses may be needed to provide the equivalent retained dose. Thus, for example, a fully retained dose of 150 kBq/kg is equivalent to a complex with a 5 day half-life administered at a dose of 711 kBq/kg. The equivalent administered dose for any appropriate retained doses may be calculated from the biological clearance rate of the complex using methods well known in the art.

In a preferable embodiment, the tissue-targeting radiopharmaceutical is administered at a dose level below the level required for a monotherapy response. This indicates a synergistic effect between the tissue-targeting radiopharmaceutical and the DNA-repair inhibitor. Preferably, the tissue-targeting radiopharmaceutical is administered at doses of greater than 10%, preferably greater than 20% less radioactivity compared to the monotherapy response (i.e. the therapy which involves administration of the tissue-targeting radiopharmaceutical only), preferably 20-50% less radioactivity compared to the monotherapy response.

Preferably, the tissue-targeting radiopharmaceutical comprises a peptide or protein tissue targeting moiety at a level of 0.02-1 mg/kg bodyweight.

Preferably the DNA-repair inhibitor is administered at a dose of 10-100 mg/kg bodyweight. In a particular embodiment the DNA-repair inhibitor may be administered over the course of at least 3 days, e.g. by following a regime cycle of 10-100 mg/kg per day twice per day for three consecutive days, followed by four days off, wherein said regime cycle is repeated four times.

The combination therapy of the present invention can be used alone or in combination with other treatment modalities including surgery, external beam radiation therapy, chemotherapy, other radionuclides, or tissue temperature adjustment etc. This forms a further, preferred embodiment of the method of the invention and formulations/medicaments may correspondingly comprise at least one additional therapeutically active agent such as another radioactive agent or a chemotherapeutic agent.

In one particular embodiment the subject is also subjected to stem cell treatment and/or other supportive therapy to reduce the effects of radium-223 induced myelotoxicity.

### Treatment/Use in Therapy

The diseased tissue to be targeted may be at a soft tissue site, at a calcified tissue site or a plurality of sites which may all be all in soft tissue, all in calcified tissue or may include at least one soft tissue site and/or at least one calcified tissue site. In one embodiment, at least one soft tissue site is targeted. The sites of targeting and the sites of origin of the disease may be the same, but alternatively may be different (such as where metastatic sites are specifically targeted). Where more than one site is involved this may include the site of origin or may be a plurality of secondary sites.

The term “soft tissue” is used herein to indicate tissues which do not have a “hard” mineralised matrix. In particular, soft tissues as used herein may be any tissues that are not skeletal tissues. Correspondingly, “soft tissue disease” as used herein indicates a disease occurring in a “soft tissue” as used herein. The invention is particularly suitable for the treatment of cancers and “soft tissue disease” thus encompasses carcinomas, sarcomas, myelomas, leukemias, lymphomas and mixed type cancers occurring in any “soft” (i.e. non-mineralised) tissue, as well as other noncancerous diseases (especially proliferative diseases) of such tissue. Cancerous “soft tissue disease” includes solid tumours occurring in soft tissues as well as metastatic and micro-metastatic tumours. Indeed, the soft tissue disease may comprise a primary solid tumour of soft tissue and at least one metastatic tumour of soft tissue in the same patient. Alternatively, the “soft tissue disease” may consist of only a primary tumour or only metastases with the primary tumour being a skeletal disease. Particularly suitable for treatment and/or targeting in all appropriate aspects of the invention are hematological neoplasms and especially neoplastic diseases of lymphoid cells, such as lymphomas and lymphoid leukemias, including Non-Hodgkin's Lymphoma, B-cell neoplasms of B-cell lymphomas. Similarly, any neoplastic diseases of bone marrow, spine (especially spinal cord) lymph nodes and/or blood cells are suitable for treatment and/or targeting in all appropriate aspects of the invention.

Some examples of B-cell neoplasms that are suitable for treatment and/or targeting in appropriate aspects of the present invention include:

Chronic lymphocytic leukemia/Small lymphocytic lymphoma, B-cell prolymphocytic leukemia, Lymphoplasmacytic lymphoma (such as Waldenstrom macroglobulinemia), Splenic marginal zone lymphoma, Plasma cell neoplasms (e.g. Plasma cell myeloma, Plasmacytoma, Monoclonal immunoglobulin deposition diseases, Heavy chain diseases), Extranodal marginal zone B cell lymphoma (MALT lymphoma), Nodal marginal zone B cell lymphoma (NMZL), Follicular lymphoma, Mantle cell lymphoma, Diffuse large B cell lymphoma, Mediastinal (thymic) large B cell lymphoma, Intravascular large B cell lymphoma, Primary effusion lymphoma and Burkitt lymphoma/leukemia.

Some examples of neoplasms suitable for treatment using a FGFR2 targeting agent of the present invention include those where mutational events are associated with tumour formation and progression including breast, endometrial and gastric cancers.

Some examples of myeloid derived neoplasms suitable for treatment using a CD33 targeted agent of the present invention includes Acute Myeloid Leukemia (AML). Some further examples of neoplasms suitable for treatment using a prostate specific membrane antigen (PSMA) targeted agent of the present invention includes prostate and brain cancers.

Some further examples of neoplasms suitable for treatment using a Human Epidermal Growth Factor Receptor-2 (HER-2) targeted agent of the present invention includes breast, gastric and ovarian cancers. Some further examples of neoplasms suitable for treatment using a mesothelin targeted agent of the present invention include malignancies such as mesothelioma, ovarian, lung and pancreatic cancer.

In a preferred embodiment the combinations of this invention are used to treat prostate cancer. The tissue-targeting radiopharmaceutical is preferably an alpha-emitting TTC which preferably comprises, but is not limited to, a monoclonal antibody targeting the tumor specific antigen PSMA.

Preferably, the combination therapy of the present invention is for the treatment of Non-Hodgkin's Lymphoma or B-cell neoplasms, breast, colorectal, endometrial, gastric, acute myeloid leukemia, prostate or brain, mesothelioma, ovarian, lung or pancreatic cancer. Typically, the combination therapy of the present invention will be used in the treatment of ovarian cancer, breast cancer, gastric cancer, lung cancer, colorectal cancer or Acute Myeloid Leukaemia.

In the combination cytotoxicity assays, the combination therapies of the present invention have been shown to have synergistic effects on the OVCAR-3 (ovarian), KATO-III (gastric), MFM-223 (breast), SUM52-PE (breast), SK-OV-3 (ovarian), BT-474 (breast), KPL-4 (breast), NCI-H226 (lung), HT29-Meso (colorectal), LNCaP-Luc (prostate) and HL-60 (Acute Myeloid Leukaemia) cancer cell lines. The in vivo efficacy studies (Ovcar-3 and MFM-223 xenograft on mice) have also shown a synergistic effect. Indeed, whilst no effect was shown for TTC alone at a dose of 100 kBq/kg dose level alone, when combined with ATR inhibitor, a significant tumor growth inhibition was observed.

### Kit

The kit of the present invention may be any kit comprising a tissue-targeting radiopharmaceutical and a DNA-repair inhibitor. The kit may comprise a container (e.g. a bottle) in which there is a mixture of the two components, or the kit may comprise two separate containers which each contain one of the two components.

### DESCRIPTION OF FIGURES

FIG. 1 shows pathways for different DNA repair mechanisms.

FIG. 2 shows an illustration of an Isobologram.

FIG. 3 shows in vitro combination cytotoxicity assay results showing the synergistic effect of MSLN-TTC+ATMi in Ovarian cancer cell line Ovcar-3.

FIG. 4 shows in vitro combination cytotoxicity assay results showing the synergistic effect of FGFR2-TTC+ATRi combination on KATO-III cancer cell line (Gastric cancer).

FIG. 5 shows in vitro combination cytotoxicity assay results showing the synergistic effect of FGFR2-TTC+ATRi combination on MFM-223 cancer cell line (Breast cancer).

FIG. 6 shows in vitro combination cytotoxicity assay results showing the synergistic effect of FGFR2-TTC+ATRi combination on SUM52PE cancer cell line (Breast cancer).

FIG. 7 shows in vitro combination cytotoxicity assay results showing the synergistic effect of Her2-TTC+ATRi combination on SK-OV-3 cancer cell line (Ovarian cancer).

FIG. 8 shows in vitro combination cytotoxicity assay results showing the synergistic effect of Her2-TTC+ATRi combination on BT-474 cancer cell line (Breast cancer).

FIG. 9 shows in vitro combination cytotoxicity assay results showing the synergistic effect of Her2-TTC+ATRi combination on KPL-4 cancer cell line (Breast cancer).

FIG. 10 shows in vitro combination cytotoxicity assay results showing the synergistic effect of MSLN-TTC+ATRi in Ovarian cancer cell line Ovcar-3.

FIG. 11 shows in vitro combination cytotoxicity assay results showing the synergistic effect of MSLN-TTC+ATRi in lung cancer cell line NCI-H226.

FIG. 12 shows in vitro combination cytotoxicity assay results showing the synergistic effect of MSLN-TTC+ATRi in colorectal cancer cell line HT29-Meso.

FIG. 13 shows in vitro combination cytotoxicity assay results showing the synergistic effect of MSLN-TTC+DNA-PKi in Ovarian cancer cell line Ovcar-3.

FIG. 14 shows in vitro combination cytotoxicity assay results showing the synergistic effect of MSLN-TTC+DNA-PKi in lung cancer cell line NCI-H226.

FIG. 15 shows in vitro combination cytotoxicity assay results showing the synergistic effect of MSLN-TTC+DNA-PKi in colorectal cancer cell lines HT29-Meso.

FIG. 16 shows in vitro combination cytotoxicity assay results showing the synergistic effect of MSLN-TTC+PARPi (Olaparib) in Ovarian cancer cell line Ovcar-3.

FIG. 17 shows in vitro combination cytotoxicity assay results showing the synergistic effect of CD33-TTC+PARPi (Olaparib) in AML cell line HL-60.

FIG. 18 shows a schematic representation of the mode of action of DNA damage sensors.

FIG. 19 shows the suppression of TTC-induced ATR kinase signalling, seen by a reduction in phosphorylated Chk1.

FIG. 20 shows a DNA histogram of cell cycle analysis showing suppression of TTC-inducedG2/M arrest by ATRi.

FIG. 21 shows the measurement of double strand DNA breaks (y-H2AX).

FIG. 22 shows the in vivo efficacy study results showing the synergistic effect of MSLN-TTC+ATRi combination on Ovcar-3 xenograft (ovarian cancer).

FIG. 23 shows the in vivo efficacy study results showing the synergistic effect of FGFR2-TTC+ATRi combination on MFM-223 (TNBC) xenograft (breast cancer).

FIG. 24 shows a histogram showing the synergistic increase in cell death by MSLN-TTC+ATRi

FIG. 25 shows cells stained for cleaved Caspase (Green fluorescence, y-axis) and y-H2AX (Red fluorescence, x-axis)

FIG. 26 shows In vitro combination cytotoxicity assay results showing the synergistic effect of PSMA-TTC+ATRi (BAY1895344) in prostate cancer cell lines LNCaP-Luc.

FIG. 27 shows in vitro combination cytotoxicity assay results showing the synergistic effect of PSMA-TTC+PARPi (Olaparib) in prostate cancer cell lines C4-2.

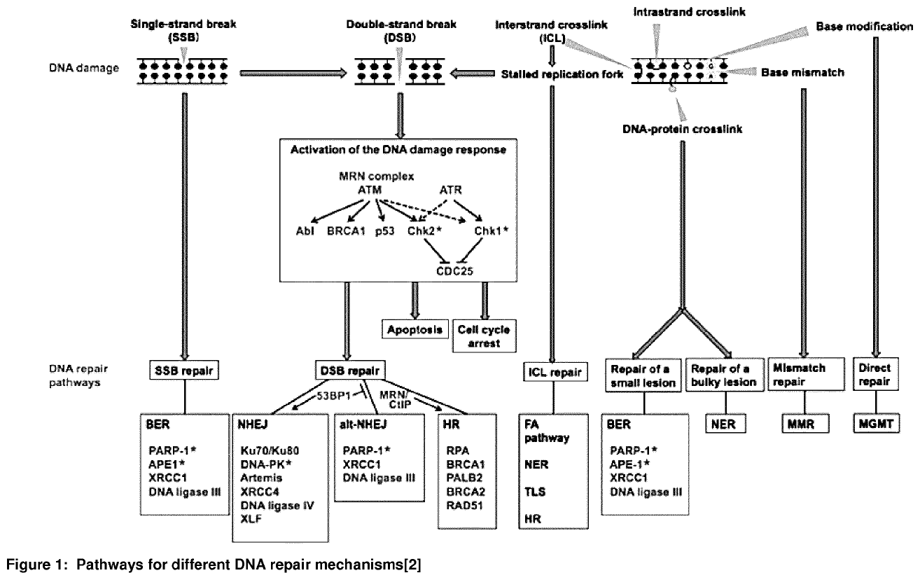

### EXAMPLES

### Example 1-Combination Cytotoxicity

Methods

The in vitro combination studies were performed with either of the two experimental methods explained:

I. Combination setup in 96 well plates:
5-20 nM inhibitor was added to cells in 96 well plate
Addition of TTC after 1 hour (titrated from 77 pM ²²⁷Th; 20 kBq/ml)
Incubated for 5-7 days
Viability determined by CellTiter-Glo (ATP); luminescence based assay
The data is plotted as % viability based on untreated control
A significant decrease in viability by the combination compared to the TTC monotreatment is defined as synergy

II. Combination setup in 384 well plates/Isobologram setup

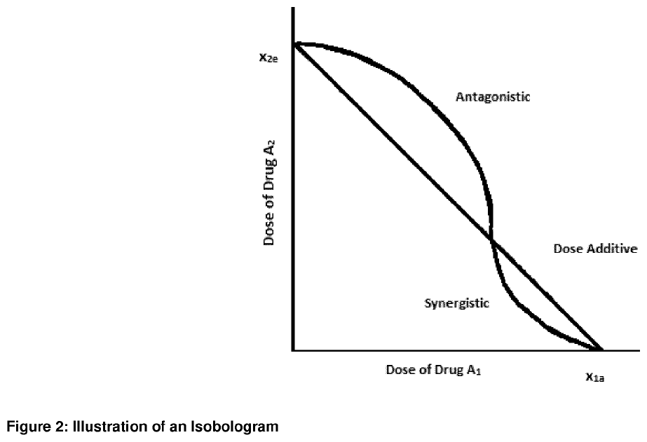

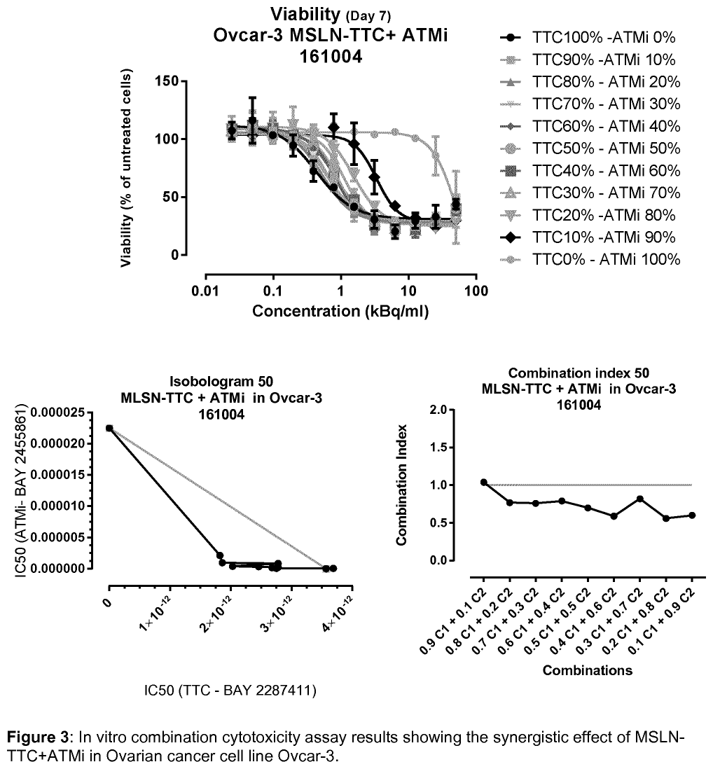

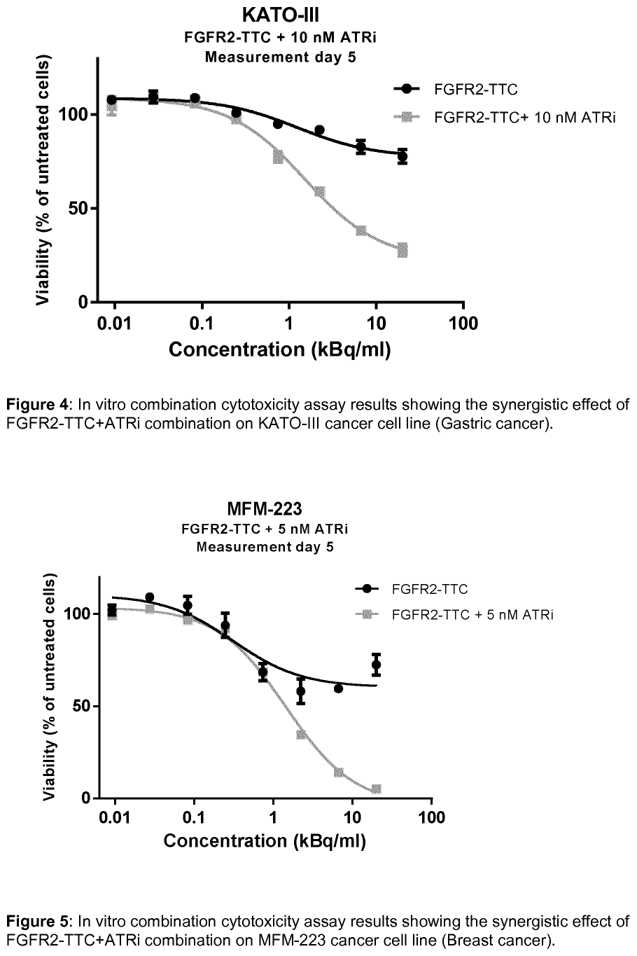

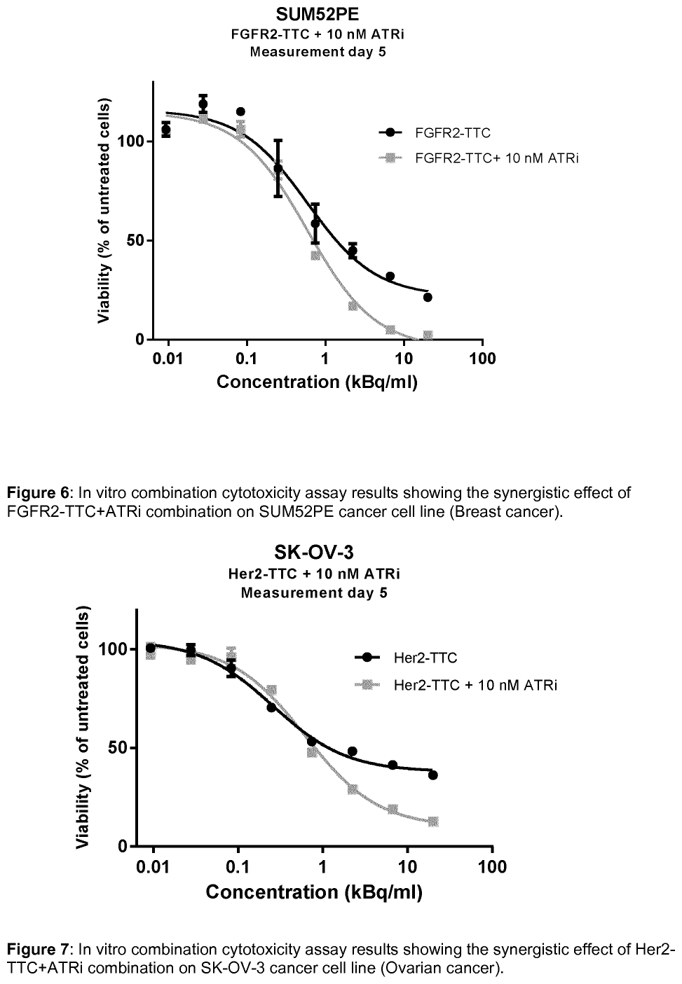

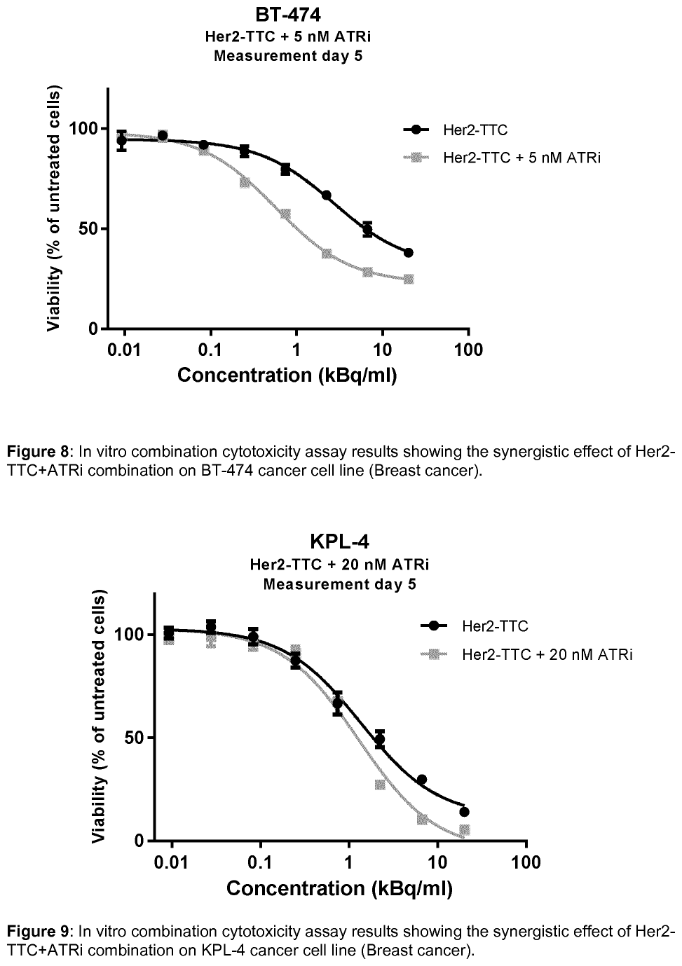

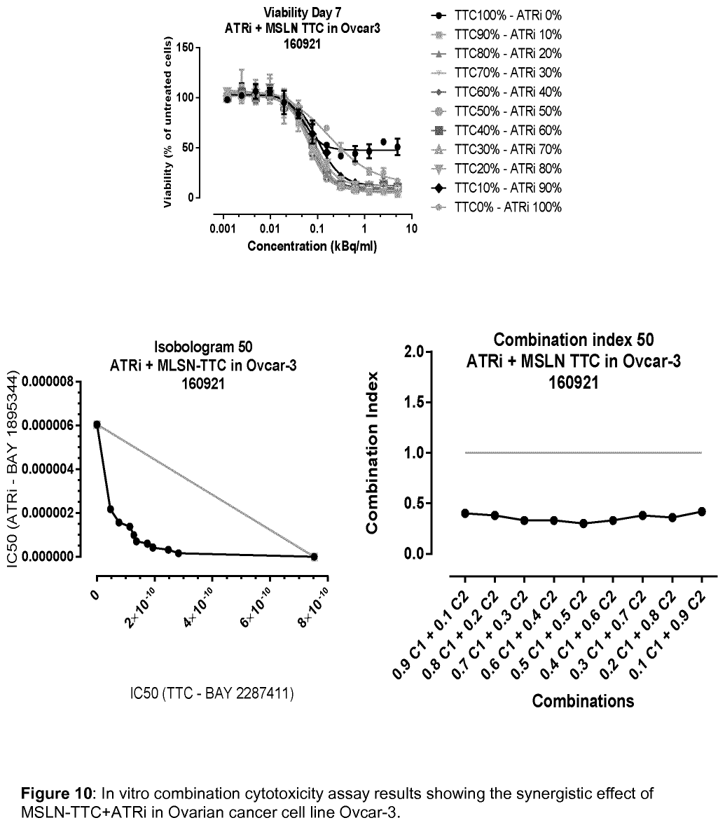

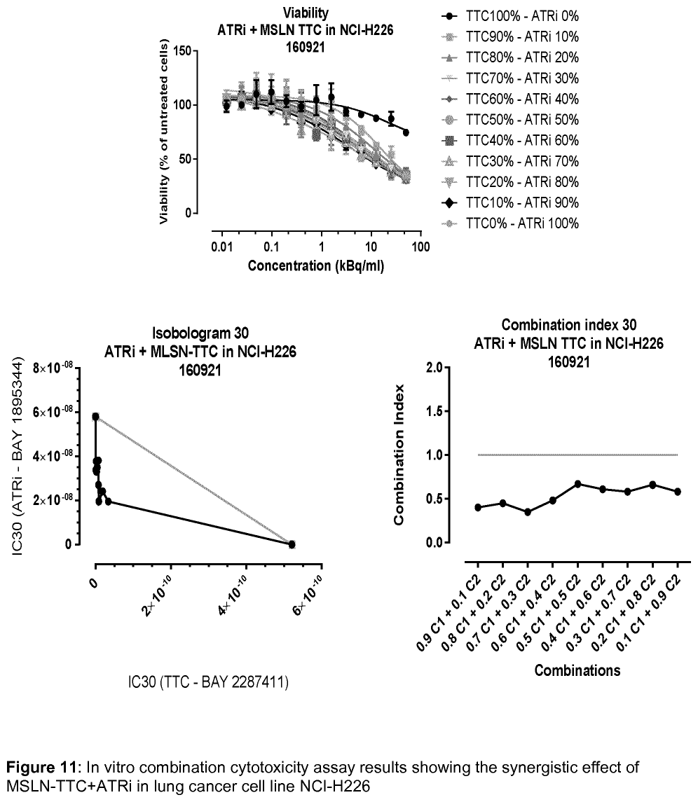

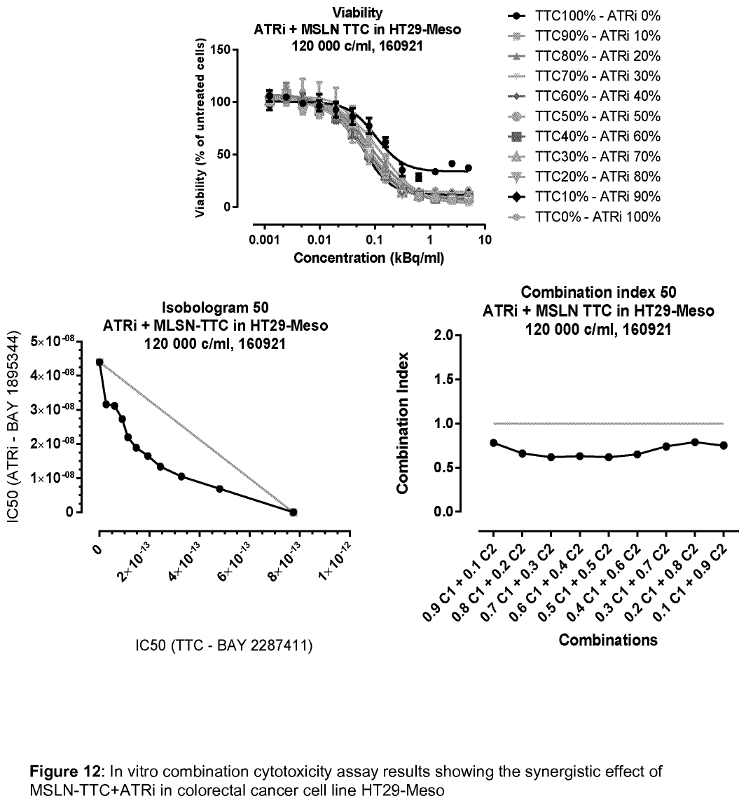

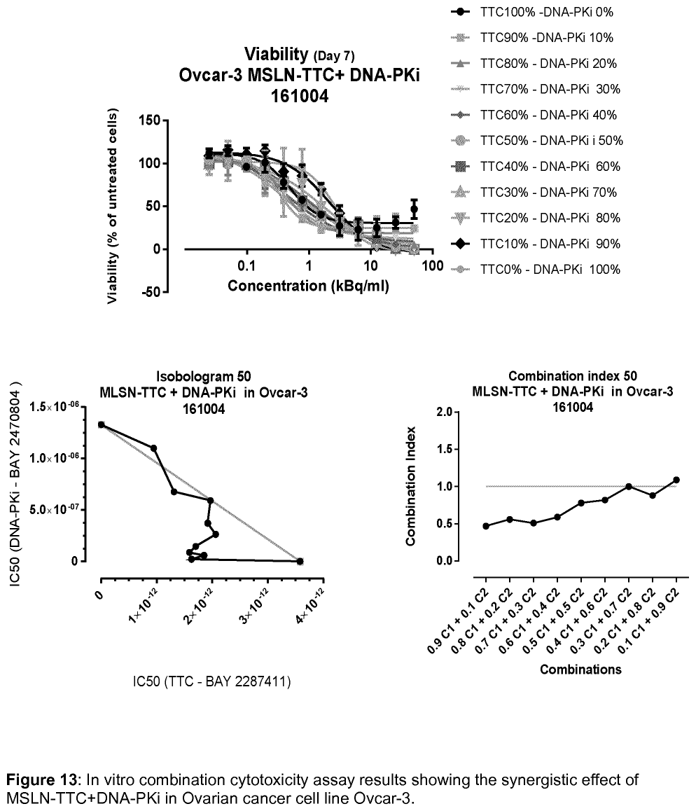

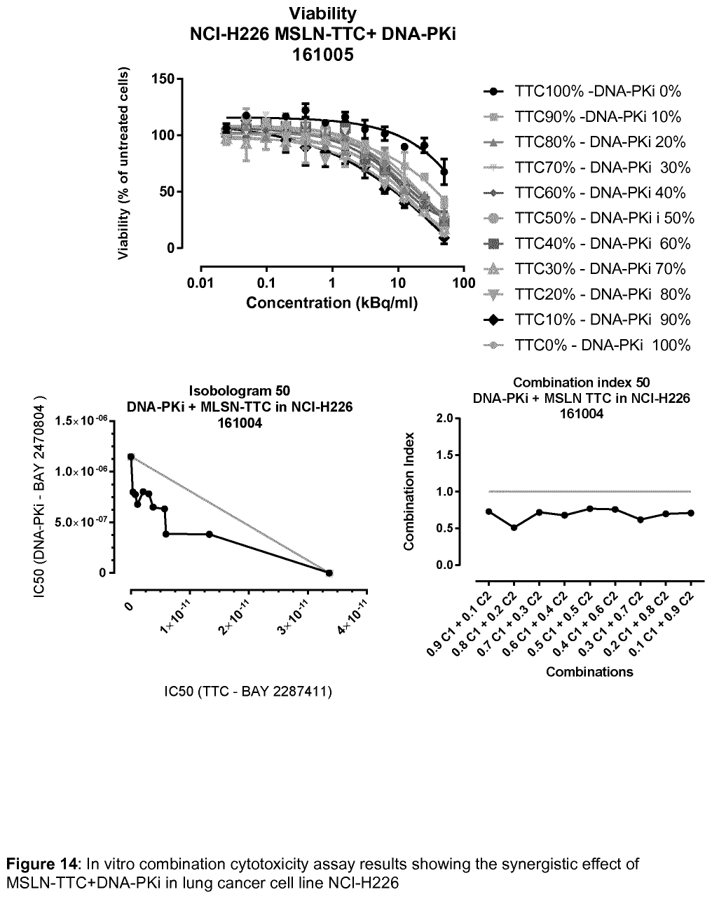

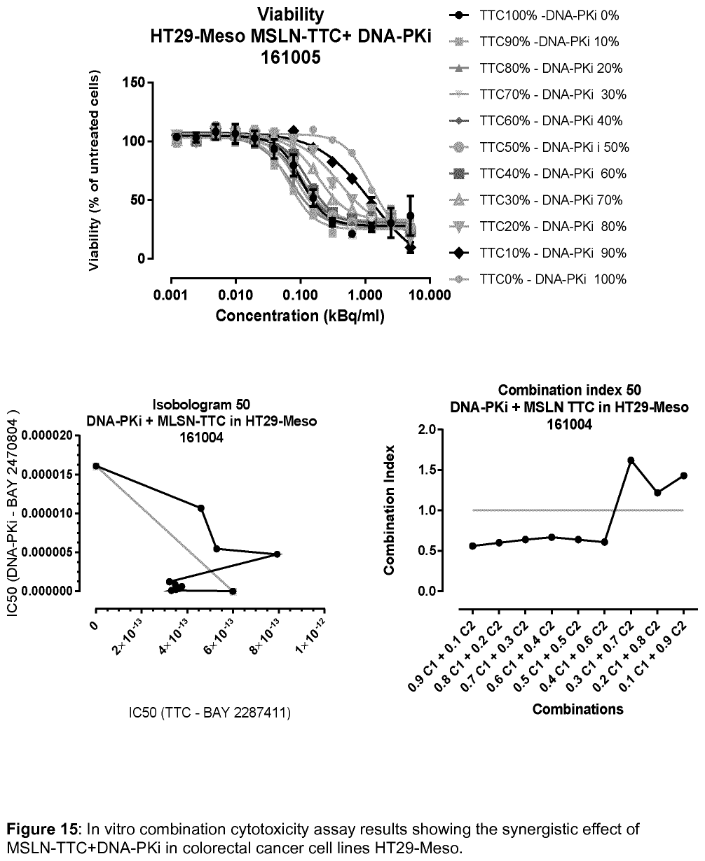

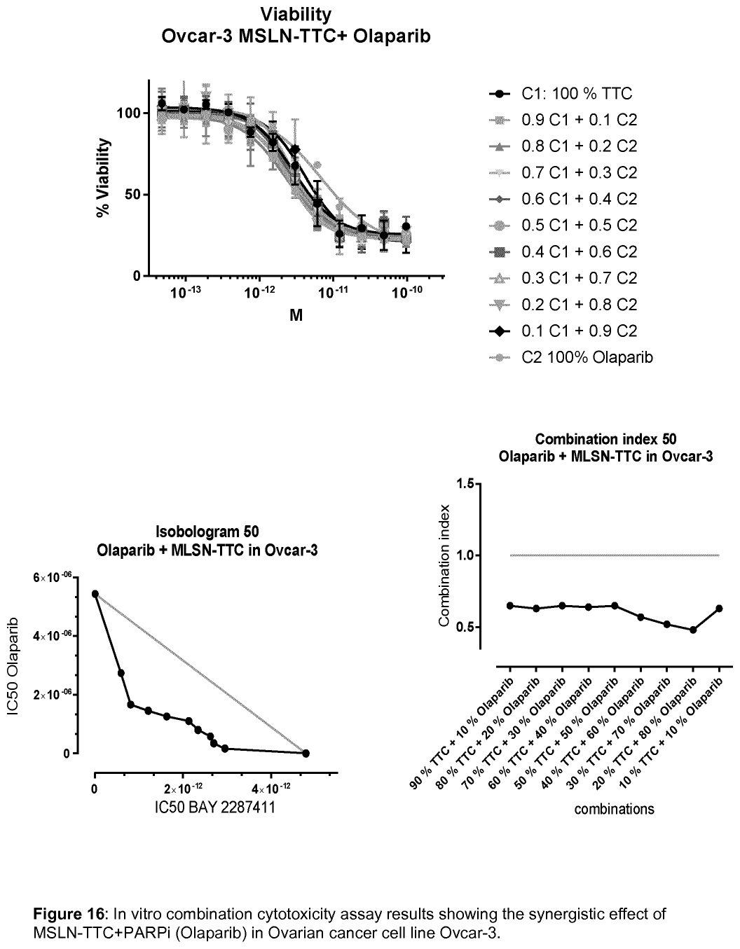

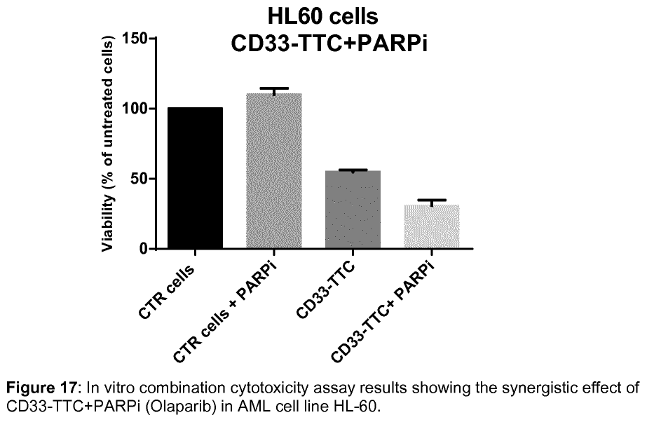

The assay evaluates the effect of the combination treatment by determining the shift in IC50 from curves established from different combination fractions [1] (see table 1).
TTC and inhibitor was added to the cells in 384 well plate
Incubated for 5-7 days
Viability determined by CellTiter-Glo (ATP); luminescence based assay
The data is plotted as % viability based on untreated control and IC50 values for the 11 curves are calculated.
The IC50 values are plotted in an isobologram, with monotreatments along the y-axis and x-axis and the IC50 values from the combinations in between these two points (see FIG. 2). If the effect is additive a straight curve will be generated between the two monotreatment-IC50 values, if the effect is synergistic the line is below the straight line and antagonistic effect gives a curve over the straight line.

III. Combination setup in 6 well plates
5 nM inhibitor was added to cells in 6 well plate
Addition of TTC after 2 hour (5-20 kBq/ml)
Incubated for 5-7 days
Viability determined by CellTiter-Glo (ATP); luminescence based assay
The data is plotted as % viability based on untreated control
A significant decrease in viability by the combination compared to the TTC monotreatment is defined as synergy

Results

A range of inhibitors have been tested in combination with TTCs in in vitro cytotoxicity assays (see table 2). The data indicates that the combination treatment results in a synergistic interaction covering a range of TTCs, inhibitor targets and cancer cell lines.

TABLE 2
Combination cytotoxicity assays

| | Small molecule | Cancer cell | Combination | |
|---|---|---|---|---|
| TTC | inhibitor | lines | Effect | FIG.(S) |
| MSLN-TTC | ATM inhibitor | OVCAR-3 | Synergistic | 3 |
| FGFR2-TTC | ATR inhibitor | KATO-III, | Synergistic | 4, 5, |
| | (BAY1895344) | MFM-223, | | 6 |
| | | SUM52-PE | | |
| Her2-TTC | ATR inhibitor | SK-OV-3, | Synergistic | 7, 8, 9 |
| | (BAY1895344) | BT-474, | | |
| | | KPL-4 | | |
| MSLN-TTC | ATR inhibitor | OVCAR3, | Synergistic | 10, 11, |
| | (BAY1895344) | NCI-H226, | | 12 |
| | | HT29-Meso | | |
| MSLN-TTC | DNA-PK | OVCAR3, | Synergistic | 13, 14, |
| | inhibitor | NCI-H226, | | 15 |
| | | HT29-Meso | | |
| MSLN-TTC | PARP inhibitor | OVCAR3 | Synergistic | 16 |
| CD33-TTC | PARP inhibitor | HL-60 | Synergistic | 17 |
| PSMA-TTC | ATR inhibitor | LNCaP-Luc | Synergistic | 26 |
| | (BAY1895344) | | | |
| PSMA-TTC | PARP inhibitor | C4-2 | Synergistic | 27 |

### Example 2-Cellular Mechanistic Assays

Methods

Cellular Mechanistic Assays

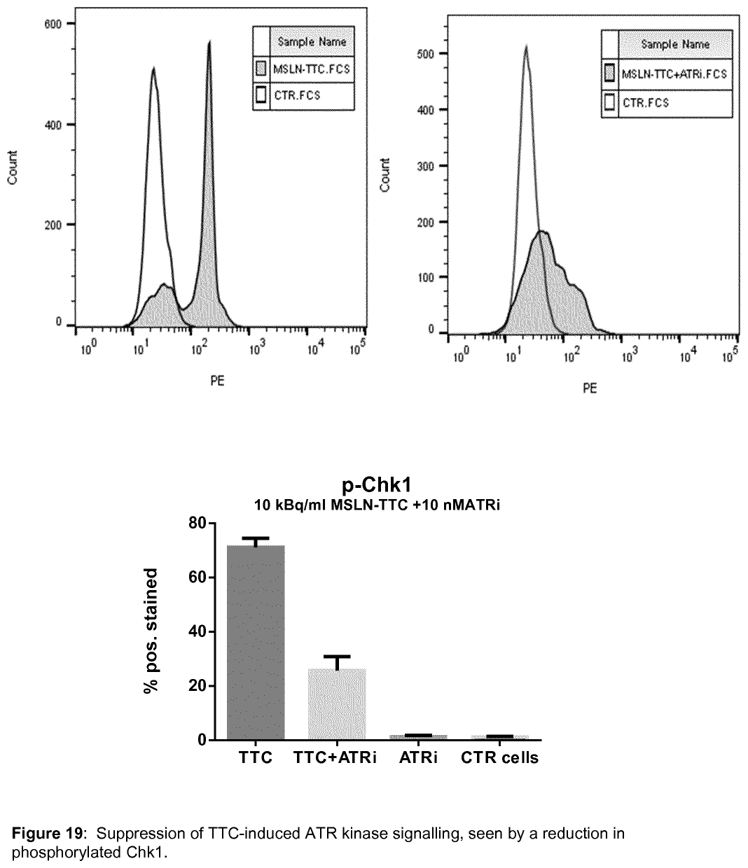

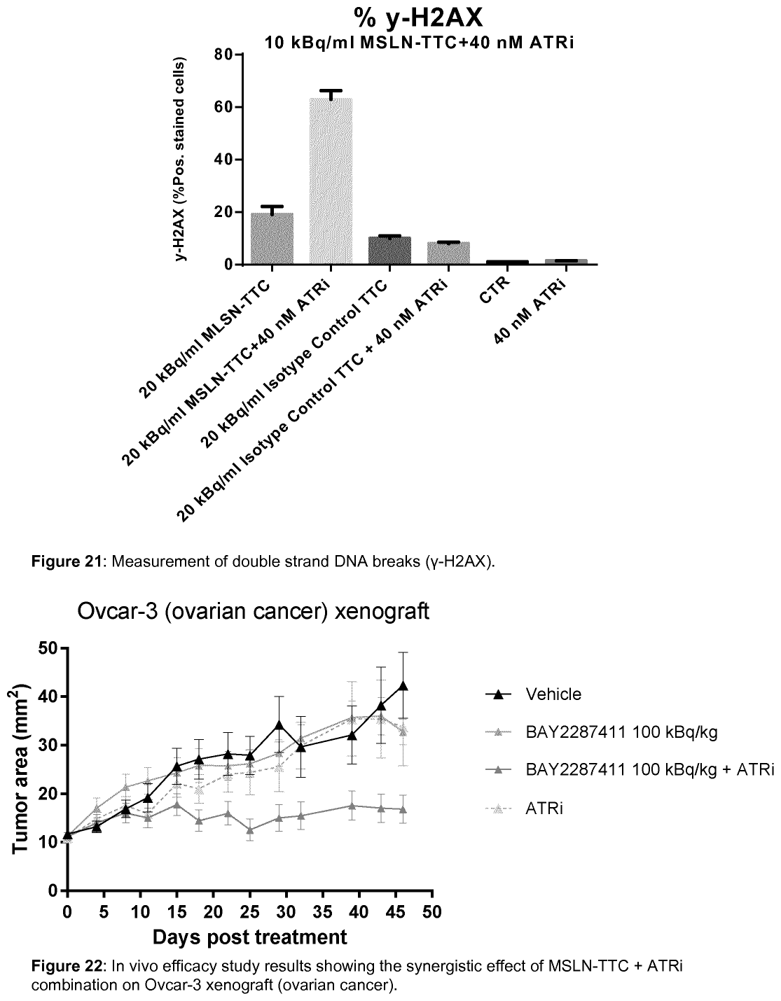

p-Chk1 (FIG. 19) and y-H2AX (FIG. 21):
Seeded cells in 6 well plates and incubated with TTC+ATRi (BAY1895344) for 3 days
Detached cells and washed two times with PBS
Cells were fixed and permeabilized cells using 70% ice cold ethanol and incubated 1 hour at 4° C.
Washed with PBS+1% FBS (flow buffer) and transfer to 96 well plate
The cells were spun down and supernatant removed
The cells were resuspended in 100 μl anti-yH2AX-A647 antibody (1:50 in flow buffer) and anti-p-Chk1 antibody (1:100 in flow buffer) and incubated for 1 hour in the dark
For cells stained with anti-p-Chk1 antibody: stained with secondary PE-antibody: 100 μl per well with Anti-rabbit IgG PE (1:100 in flow buffer) and incubated in dark for 1 hour at 4° C.
Washed two times with flow buffer and removed the supernatant
Resuspended the cells in 200 μl flow buffer and transferred to a u-shaped 96 well plate
The plate was analysed by columns on the EasyCyte 8HT (log scale, medium flow rate).

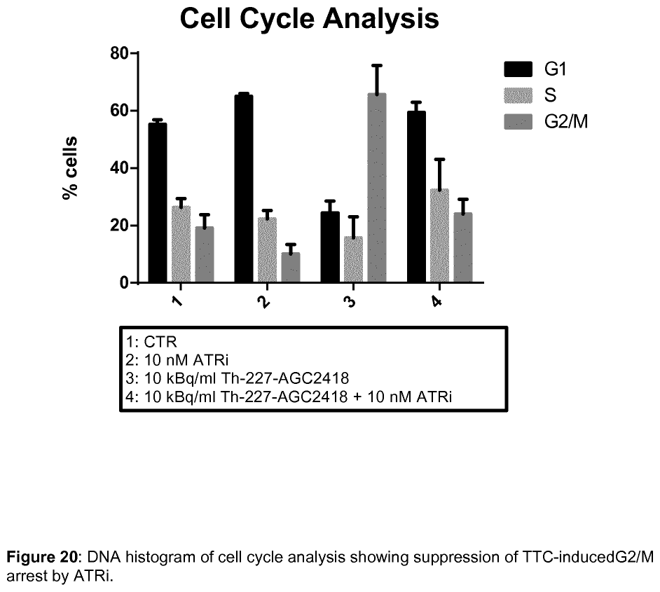

Cell cycle analysis (DNA histogram—FIG. 20):
Seeded cells in 6 well plates and incubated with TTC+ATRi (BAY1895344) for 3 days
Detached cells and washed two times with PBS
Fixed and permeabilized cells using 70% ice cold ethanol and incubate 1 hour at 4° C.
Washed cells with PBS+1% FBS and transfer to 96 well plate
The cells were spun down and supernatant removed
Resuspend the cells in 100 μl PI/RNase and incubated for 30 minutes in the dark at 4° C.
Analyse the plate by columns on the EasyCyte 8HT (linear scale, low flow rate).

Results

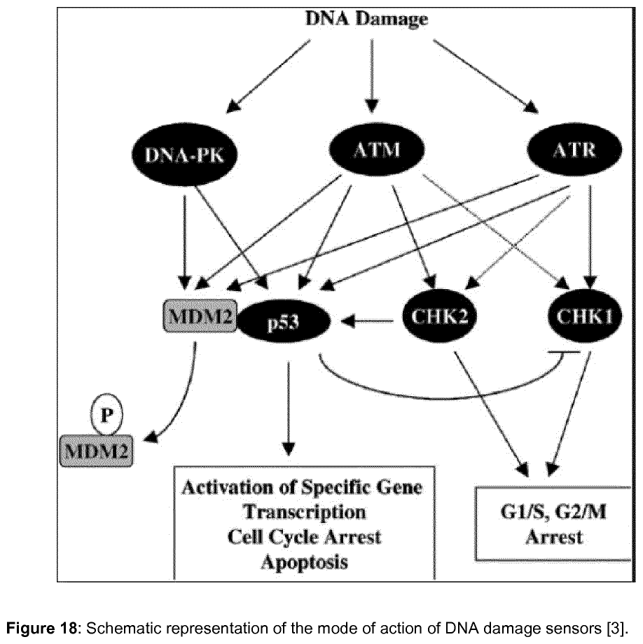

A schematic representation of the mode of action of DNA damage sensors is shown in FIG. 18. The mechanism of action for the combination of TTC and ATRi (BAY1895344) was explored by performing different experiments, including measurement of phosphorylated Chk1 (FIG. 18), cell cycle analysis (FIG. 19) and measurements of double strand DNA breaks (y-H2AX, FIG. 20). In short the data indicates that the combination with ATR inhibitor:
Suppress TTC-induced ATR kinase signaling, seen by a reduction in phosphorylated Chk1
Suppress TTC induced G2-cell cycle arrest, seen by a shift in cell cycle distribution
Suppress repair of double strand DNA break, seen by a higher degree of double strand DNA breaks compared to TTC monotreatment

Ultimately this leads to increased cell death by the combination treatment compared to the monotreatment. This can be explained by accumulation of DNA damage leading to mitotic catastrophe.

### Example 3-In vivo, Efficacy Studies

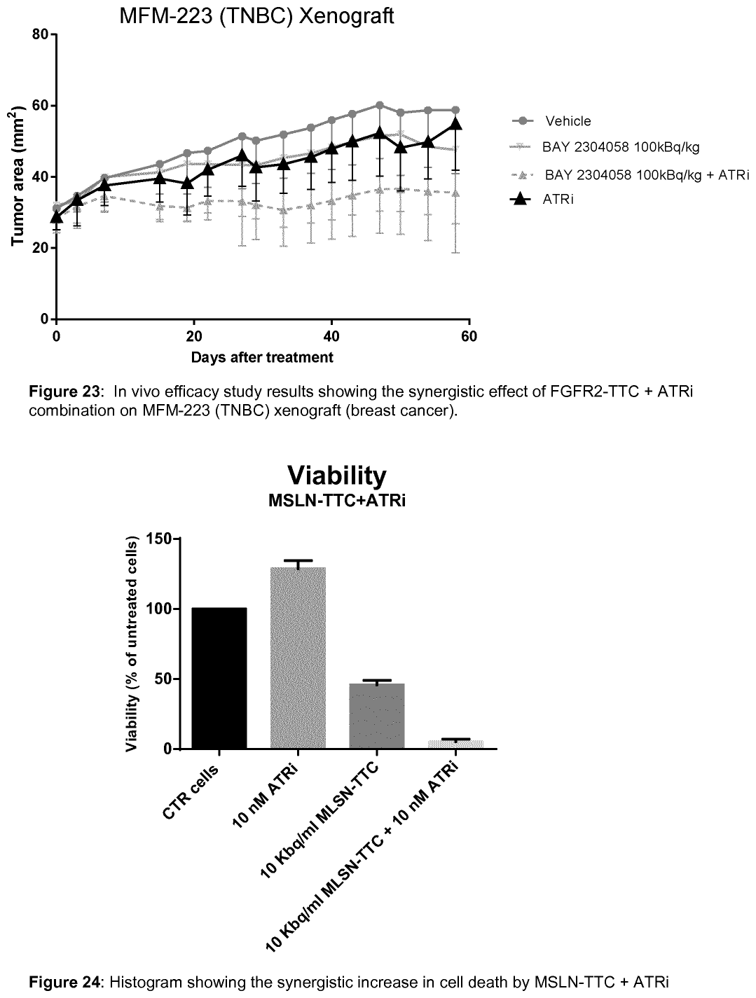

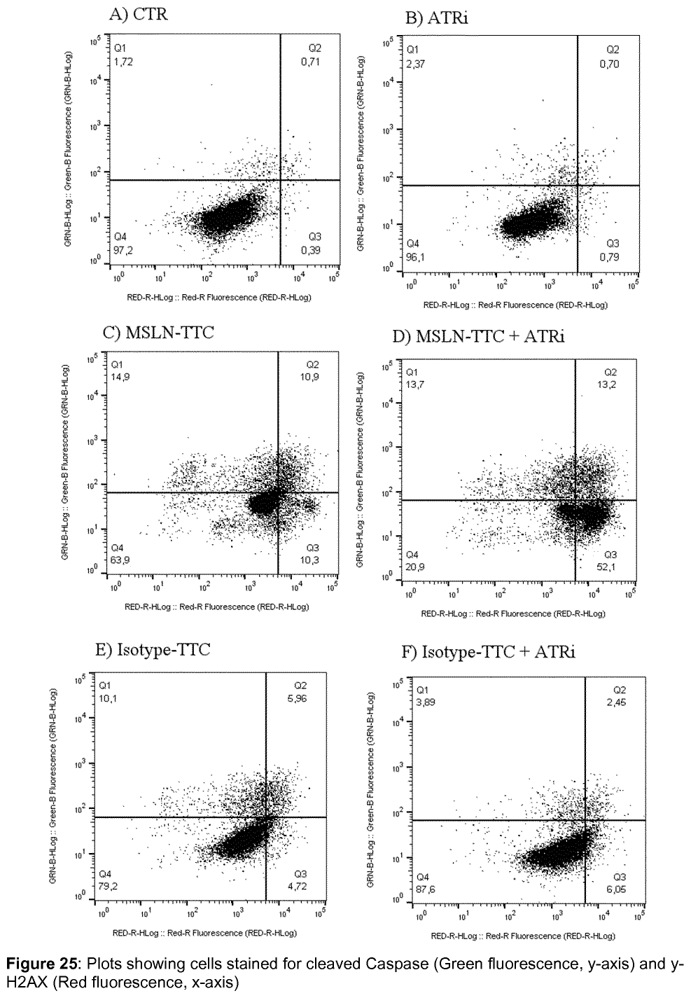

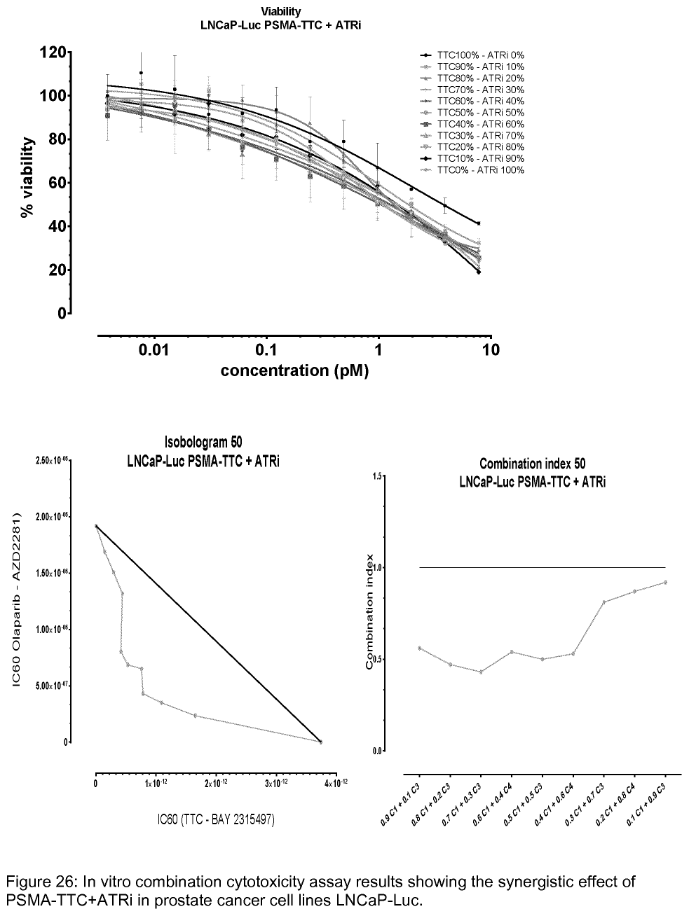

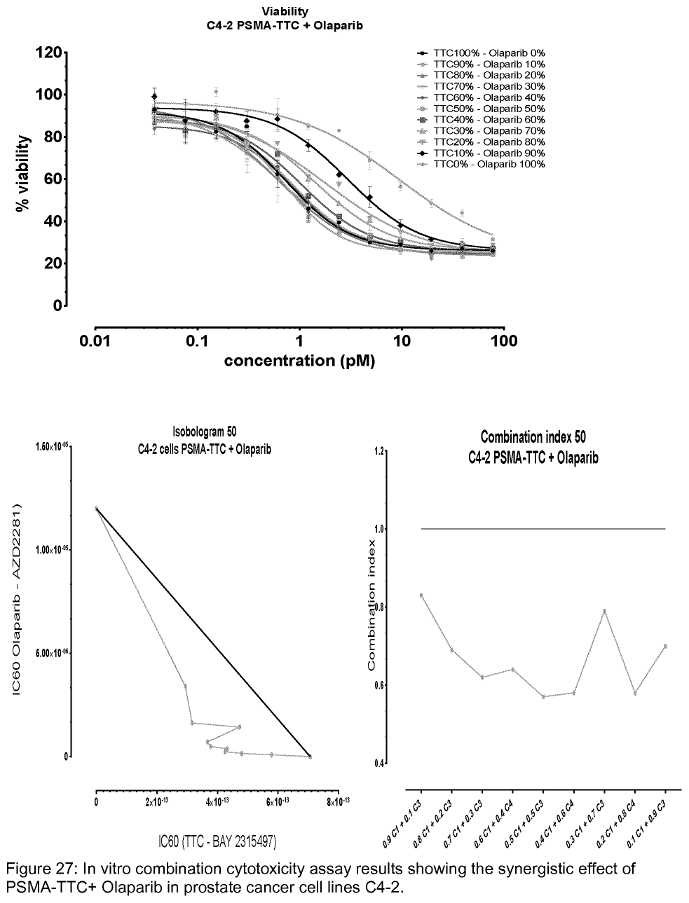

The combination of TTC and ATRi (BAY1895344) was also evaluated in in vivo efficacy studies. Two different xenograft models were evaluated:
Ovcar-3 xenograft in nude mice (FIG. 22)—MSLN-positive ovarian cancer cell line, treated with MSLN-TTC in combination with ATRi (BAY1895344)
MFM-223 xenograft in nude mice (FIG. 23)—FGFR2-positive breast cancer cell line, treated with FGFR2-TTC in combination with ATRi (BAY1895344)

Methods

Ovcar-3 xenograft model (FIG. 22):
At study day 0, animals received a subcutaneous inoculation of 5×10⁶ humane ovarian Ovcar-3 cells/mouse on the right flank.
Upon reaching a palpable tumor size (20-25 mm²), test item MSLN-TTC (BAY2287411) was injected into the tail vein of the animals at 100 kBq/kg with a protein dose of 0.14 mg/kg.
After initial dosing of MSLN-TTC the ATRi (BAY 1895344) was dosed orally in a cycle of 20 mg/kg twice per day in a row of three days, followed by 4 days off. The first treatment started 7 days after MSLN-TTC had been given and in total 4 cycles of ATRi were given.
The tumor growth and the body weights were measured every other or third day. Upon reaching the humane endpoint, tumor volume >1500 mm³ or largest diameter of 15 mm, animals will euthanized upon cervical dislocation. Animals will be assessed for any major toxicological signs during necropsy. Major organs (including liver, lung, kidney, spleen and bone marrow) as well as organs with any observed abnormalities will be harvested, fixed and processed to histopathology to assess for histopathological changes due to treatment.

MFM-223 xenograft model (FIG. 23):
At study day 0, animals received an orthotopic inoculation of 2.5×10⁶ MFM-223 cells/mouse into the upper right mammary fat pad.
Upon reaching a palpable tumor size (30-35 mm²), test item FGFR2-TTC (BAY2304058) was injected into the tail vein of the animals at 100 kBq/kg with a protein dose of 0.14 mg/kg.
After initial dosing of FGFR2-TTC the ATRi (BAY 1895344) was dosed orally in a cycle of 40 mg/kg twice per day in a row of three days, followed by 4 days off. The first treatment started 7 days after FGFR2-TTC had been given and in total 4 cycles of ATRi (BAY1895344) were given.
The tumor growth and the body weights were measured every other day, on Monday, Wednesday and Friday. Upon reaching the humane endpoint, tumor volume >1500 mm³ or largest diameter of 15 mm, animals were euthanized upon cervical dislocation. Animals were assessed for any major toxicological signs during necropsy. Major organs (including liver, lung, kidney, spleen and bone marrow) as well as organs with any observed abnormalities will be harvested, fixed and processed to histopathology to assess for histopathological changes due to treatment.

Results

Both studies indicated that there was a synergistic effect by the combination of TTC and ATRi (BAY1895344). While no effect was shown for 100 kBq/kg dose level alone, when combined with ATR inhibitor, a significant tumor growth inhibition was observed.

### REFERENCES

1. Tallarida, R. J., An overview of drug combination analysis with isobolograms. J Pharmacol Exp Ther, 2006. 319(1): p. 1-7.

2. Hosoya, N. and K. Miyagawa, Targeting DNA damage response in cancer therapy. Cancer Sci, 2014. 105(4): p. 370-88.

3. Yang, J., Y. Yu, and P. J. Duerksen-Hughes, Protein kinases and their involvement in the cellular responses to genotoxic stress. Mutat Res, 2003. 543(1): p. 31-58.

1. A method of combination therapy, comprising administering
a) a tissue-targeting radiopharmaceutical, and
b) a DNA-repair inhibitor.

2. The method of claim 1, wherein the tissue-targeting radiopharmaceutical comprises an alpha-emitter.

3. The method of claim 1, wherein the tissue-targeting radiopharmaceutical is a complex comprising the 4+ ion of an alpha-emitting thorium radionuclide such as Thorium-227.

4. The method of claim 1, wherein the tissue-targeting radiopharmaceutical is a targeted thorium conjugate (TTC).

5. The method of claim 1, wherein the tissue-targeting radiopharmaceutical comprises a tissue-targeting moiety selected from a monoclonal or polyclonal antibody, an antibody fragment (such as Fab, F(ab′)2, Fab′ or scFv), a construct of such antibodies and/or fragments, a protein, a peptide or a peptidomimetic.

6. The method of claim 1, wherein the tissue-targeting radiopharmaceutical comprises a tissue-targeting moiety which has binding affinity for the CD22 receptor, FGFR2, Mesothelin, HER-2, PSMA or CD33.

7. The method of claim 1, wherein the DNA-repair inhibitor is an inhibitor of a protein selected from the group consisting of PARP1, ATR, ATM and DNA-PK.

8. The method of claim 1, wherein the DNA-repair inhibitor is selected from the group consisting of BAY1895344, olaparib, AZD0156 and VX984.

9. The method of claim 1, wherein the DNA-repair inhibitor is selected from a PI3k inhibitor, an EGFR inhibitor and/or antibody, an AKT inhibitor, an mTOR inhibitor, an MEK inhibitor, a WEE1 inhibitor, a Chk1 and/or Chk2 inhibitor, or a RAD51 inhibitor.

10. claim for the treatment of hyperplastic or neoplastic disease, The method of claim 1, for treatment of a hyperplastic or neoplastic disease in an animal in need thereof, comprising administering to the animal effective amounts of the tissue-targeting radiopharmaceutical and the DNA-repair inhibitor.

11. The method of claim 1, wherein the tissue-targeting radiopharmaceutical is administered at a dose level below the level required for a monotherapy response.

12. The method of claim 1, wherein the tissue-targeting radiopharmaceutical and the DNA-repair inhibitor are administered sequentially in either order.

13. The method of claim 1, wherein the tissue-targeting radiopharmaceutical is administered before the DNA-repair inhibitor.

14. The method of claim 1, wherein the DNA-repair inhibitor is administered at least 2 days after administration of the tissue-targeting radiopharmaceutical.

15. The method of claim 1, wherein the tissue-targeting radiopharmaceutical is administered at a dose of 20-200 kBq/kg.

16. The method of claim 1, wherein the tissue-targeting radiopharmaceutical comprises a peptide or protein tissue targeting moiety at a level of 0.02-1 mg/kg.

17. The method of claim 1, wherein the DNA-repair inhibitor is administered at a dose of 10-100 mg/kg.

18. The method of claim 1, wherein the DNA-repair inhibitor is administered over the course of at least 3 days.

19. The method of claim 10, comprising administering
a) the tissue-targeting radiopharmaceutical, and
b) the DNA-repair inhibitor,
simultaneously or sequentially in either order.

20. (canceled)

21. (canceled)

22. (canceled)

23. (canceled)

24. A kit containing a tissue-targeting radiopharmaceutical and a DNA-repair inhibitor for simultaneous, separate or sequential use in the treatment of a hyperplastic or neoplastic disease.

25. (canceled)

26. (Canceled)

27. A kit comprising:
a) a tissue-targeting radiopharmaceutical, and
b) a DNA-repair inhibitor.

28. The method of claim 6, wherein the tissue-targeting radiopharmaceutical comprises a tissue-targeting moiety which has binding affinity for Mesothelin, FGFR2, HER-2 or CD33.

29. The method of claim 7, wherein the DNA-repair inhibitor is an inhibitor of ATR.

30. The method of claim 9, wherein the DNA-repair inhibitor is a PI3k inhibitor or an EGFR inhibitor and/or antibody.

31. The method of claim 10, wherein the hyperplastic or neoplastic disease is a carcinoma, sarcoma, myeloma, leukemia, lymphoma, or mixed type cancer.

32. The method of claim 31, wherein the hyperplastic or neoplastic disease is Non-Hodgkin's Lymphoma, B-cell neoplasms, breast cancer, colorectal cancer, endometrial cancer, gastric cancer, acute myeloid leukemia, prostate cancer, brain cancer, mesothelioma, ovarian cancer, lung cancer or pancreatic cancer.

33. The kit according to claim 24, wherein the hyperplastic or neoplastic disease is a carcinoma, sarcoma, myeloma, leukemia, lymphoma, or mixed type cancer.

34. The kit according to claim 33, wherein the hyperplastic or neoplastic disease is Non-Hodgkin's Lymphoma, B-cell neoplasms, breast cancer, colorectal cancer, endometrial cancer, gastric cancer, acute myeloid leukemia, prostate cancer, brain cancer, mesothelioma, ovarian cancer, lung cancer or pancreatic cancer.
